(19) United States
(12) Patent Application Publication
(10) Pub. No.: US 2025/0229104 A1
(43) Pub. Date: Jul. 17, 2025

(54) AUTOMATED GENERATION OF RADIOTHERAPY PLANS

(71) Applicants: MEMORIAL SLOAN-KETTERING CANCER CENTER; MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Masoud Zarepisheh, New York, NY (US); Saad Nadeem, New York, NY (US)

(21) Appl. No.: 18/854,778

(22) PCT Filed: Apr. 6, 2023
(86) PCT No.: PCT/US2023/017786

§ 371 Date: Oct. 7, 2024

Related U.S. Application Data
(60) Provisional application No. 63/329,180, filed on Apr. 8, 2022.

(51) Int. Cl. A61N 5/10; G06N 20/00; G16H 20/40; G16H 30/40; G16H 50/70

(52) U.S. Cl. CPC A61N 5/103; G06N 20/00; G16H 20/40; G16H 30/40; G16H 50/70; A61N 2005/1041

(57) ABSTRACT

Presented herein are systems, methods, and non-transient computer readable media for determining radiation therapy dosages to administer. A computing system may identify a first dataset comprising: (i) a biomedical image derived from a first sample to be administered with radiotherapy and (ii) an identifier corresponding to an organ from which the first sample is obtained. The computing system may apply, to the first dataset, a machine learning (ML) model comprising a plurality of weights trained using a plurality of second datasets in accordance with a moment loss for each of a plurality of organs. The computing system may determine, from applying the first dataset to the ML model, a radiation therapy dose to administer to the sample from which the biomedical image is derived. The computing system may store an association between the first dataset and the radiation therapy dose.

### CROSS REFERENCE TO RELATED APPLICATIONS

The present application claims priority to U.S. Provisional Patent Application No. 63/329,180, titled “ARTIFICIAL INTELLIGENCE (AI) GUIDED RAPID DOSE PLANNING,” filed Apr. 8, 2022, which is incorporated by reference in its entirety.

### BACKGROUND

A computing system may apply various machine learning (ML) techniques to an input to generate an output.

### SUMMARY

Aspects of the present disclosure are directed to systems, methods, and non-transient computer readable media for determining radiation therapy dosages to administer. A computing system may identify a first dataset comprising (i) a first biomedical image derived from a first sample to be administered with radiotherapy and (ii) a first identifier corresponding to a first organ of a plurality of organs from which the first sample is obtained. The computing system may apply, to the first dataset, a machine learning (ML) model comprising a plurality of weights trained using a plurality of second datasets in accordance with a moment loss for each of the plurality of organs. Each of the plurality of second datasets may include (i) a respective second biomedical image derived from a second sample, (ii) a respective second identifier corresponding to a second organ of the plurality of organs from which the second sample is obtained, and (iii) a respective annotation identifying a corresponding radiation therapy dose to administer to the second sample. The computing system may determine, from applying the first dataset to the ML model, a radiation therapy dose to administer to the sample from which the first biomedical image is derived. The computing system may store, using one or more data structures, an association between the first dataset and the radiation therapy dose.

In some embodiments, the computing system may provide information for the radiotherapy to administer on the sample based on the association between the first dataset and the radiation therapy dose. In some embodiments, the computing system may generate a radiotherapy plan to administer via a radiotherapy device to the first sample using the association between the first dataset and the radiation therapy dose.

In some embodiments, the computing system may receive a plurality of first datasets corresponding to a plurality of samples from one or more of the plurality of organs of the subject. In some embodiments, the computing system may determine a plurality of radiation therapy doses to administer to the corresponding plurality of samples from one or more of the plurality of organs of the subject. In some embodiments, the computing system may generate a radiotherapy plan to administer via a radiotherapy device to the subject based on the plurality of radiation therapy doses.

In some embodiments, the computing system may determine, from applying the first dataset to the ML model, at least one of a mean radiation therapy dose or a maximum radiation therapy dose based on the first organ. In some embodiments, the first biomedical image may include a first tomogram with a mask identifying a condition in a portion of the sample to be addressed via administration of the radiotherapy dose.

In some embodiments, the computing system may determine, from applying the first dataset to the ML model, a plurality of parameters for the radiation therapy dose comprising one or more of (i) an identification of a portion of the sample to be administered with the radiotherapy dose; (ii) an intensity of a beam to be applied on the first sample; (iii) a shape of the beam; (iv) a direction of a beam relative to the first sample; and (v) a duration of application of the beam on the first sample.

Aspects of the present disclosure are directed to the systems, methods, and non-transient computer readable media for training models to determine radiation therapy dosages to administer. A computing system may identify a plurality of datasets, each comprising (i) a respective biomedical image derived from a corresponding sample, (ii) a respective identifier corresponding to a respective organ of a plurality of organs from which the corresponding sample is obtained, and (iii) a respective annotation identifying a corresponding first radiation therapy dose to administer to the sample. The computing system may apply, to the plurality of datasets, a machine learning (ML) model comprising a plurality of weights to determine a plurality of second radiation therapy doses to administer. The computing system may generate at least one moment loss for each organ of the plurality of organs based on a comparison between (i) a subset of the plurality of second radiation therapy for the organ and (ii) a corresponding set of first radiation therapy doses from a subset of the plurality of datasets, each comprising the respective identified corresponding to the organ. The computing system may one or more of the plurality of weights of the ML model in accordance with the at least one moment loss for each organ of the plurality of organs.

In some embodiments, the computing system may generate a voxel loss based on (i) a second radiation therapy dose of the plurality of second radiation therapy doses and (ii) the corresponding first radiation therapy dose identified in the annotation. In some embodiments, the computing system may modify one or more of the plurality of weights of the ML model in accordance with a combination of the at least one moment loss for each organ and the voxel loss across the plurality of datasets.

In some embodiments, the computing system may generate the at least one moment loss further based on a set of voxels identified for the organ within the respective biomedical image in at least one of the plurality of datasets. In some embodiments, the ML model comprises the plurality of weights arranged in accordance with an encoder-decoder model to determine each of the plurality of second radiation therapy doses to administer using a corresponding dataset of the plurality of datasets. In some embodiments, the computing system may determine, from applying a dataset of the plurality of datasets to the ML model, at least one of a mean radiation therapy dose or a maximum radiation therapy dose based on the organ identified in the dataset.

In some embodiments, the first radiation therapy dose and the second radiation therapy dose each comprise one or more of (i) an identification of a portion of the respective sample to be administered; (ii) an intensity of a beam to be applied; (iii) a shape of the beam; (iv) a direction of a beam, and (v) a duration of application of the beam. In some embodiments, the respective biomedical image in each of the plurality of datasets further comprises a respective tomogram with a mask identifying a condition in a portion of the respective sample to be addressed via administration of the radiotherapy dose.

### BRIEF DESCRIPTION OF THE DRAWINGS

FIG. 1: Entire process of training a 3D CNN network to generate a 3D voxelwise dose. OARs are one-hot encoded and concatenated along the channel axis with CT, PTV and FCBB beam dose as input to the network.

FIG. 2: A 3D Unet-like CNN architecture used to predict 3D voxelwise dose.

FIG. 3: Boxplots illustrating the statistics of OpenKBP dose and DVH scores for all 20 test datasets using different inputs, loss functions, and training datasets.

FIG. 4: An example of predicted doses using CT with OAR/PTV only, CT with OAR/PTV/Beam with MAE loss and CT with OAR/PTV/Beam with MAE+DVH loss as inputs to the CNN.

FIG. 5: Overview of data processing pipeline and training a 3D network to generate a 3D voxelwise dose. OARs are one-hot encoded and concatenated along the channel axis with CT and PTV input to the network.

FIG. 6: 3D Unet architecture used to predict 3D voxelwise dose.

FIG. 7: Left) DVH-score and dose-score comparison for (i) MAE loss, (ii) MAE+DVH loss, and (iii) MAE+Moment loss. Right) Time comparison for training UNET using (i) MAE loss, (ii) MAE+DVH loss, and (iii) MAE+Moment loss.

FIG. 8: Absolute Error (in percentage of prescription) for max/mean dose for organ-at-risk and PTV D95/D99 dose. The lower is always the better.

FIG. 9: DVH plots for different structures using i) Actual Dose, predicted dose using ii) MAE loss, iii) MAE+DVH loss, and iv) MAE+Moment loss.

FIG. 10: Absolute Error (in percentage of prescription) for max/mean dose for organ-at-risk and PTV D95/D99 dose. The lower is always the better.

FIG. 11 depicts a block diagram of a system for determining radiation therapy dosages to administer in accordance with an illustrative embodiment.

FIG. 12 depicts a block diagram of a process to train models in the system for determining radiation therapy dosages to administer in accordance with an illustrative embodiment.

FIG. 13 depicts a block diagram of a process for evaluating acquired images in the system for determining radiation therapy dosages to administer in accordance with an illustrative embodiment.

FIG. 14 depicts a flow diagram of a method of training models to determine radiation therapy dosages in accordance with an illustrative embodiment.

FIG. 15 depicts a flow diagram of a method of determining radiation therapy dosages in accordance with an illustrative embodiment.

FIG. 16 depicts a block diagram of a server system and a client computer system in accordance with an illustrative embodiment.

### DETAILED DESCRIPTION

Following below are more detailed descriptions of various concepts related to, and embodiments of, systems and methods for artificial intelligence (AI) guided dose planning. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes deep learning 3D dose prediction for lung intensity modulated radiation therapy (IMRT) using consistent or unbiased automated plans.

Section B describes domain knowledge-driven 3D dose prediction using moment-based loss function.

Section C describes systems and methods of determining radiation therapy dosages to administer to subjects.

Section D describes a network environment and computing environment which may be useful for practicing various embodiments described herein.

### A. Deep Learning 3D Dose Prediction for Lung Intensity Modulated Radiation Therapy (IMRT) Using Consistent or Unbiased Automated Plans

### 1. Introduction

Deep learning (DL) may be used to perform 3D dose prediction. However, the variability of plan quality in the training dataset, generated manually by planners with a wide range of expertise, can dramatically affect the quality of the final predictions. Moreover, any changes in the clinical criteria requires a new set of manually generated plans by planners to build a new prediction model. In the present disclosure, consistent plans generated by the in-house automated planning system (named “ECHO”) may be used to train the DL model. ECHO (expedited constrained hierarchical optimization) generates consistent and unbiased plans by solving large-scale constrained optimization problems sequentially. If the clinical criteria changes, a new training data set can be easily generated offline using ECHO, with no or limited human intervention, making the DL-based prediction model easily adaptable to the changes in the clinical practice. 120 conventional lung patients (100 for training, 20 for testing) with different beam configurations may be used to train the DL-model using manually-generated plans as well as automated ECHO plans. Different inputs may be evaluated: (1) CT+ (PTV/OAR) contours, and (2) CT+contours+beam configurations, and different loss functions: (1) MAE (mean absolute error), and (2) MAE+DVH (dose volume histograms). The quality of the predictions was compared using different DVH metrics as well as dose-score and DVH-score. The best results were obtained using automated ECHO plans and CT+contours+beam as training inputs and MAE+DVH as loss function

Despite advances in optimization and treatment planning, intensity modulated radiation therapy (IMRT) treatment planning remains a time-consuming and resource-demanding task with the plan quality heavily dependent on the planner's experience and expertise. This problem is even more pronounced for challenging clinical cases such as conventional lung with complex geometries and intense conflict between the objectives of irradiating planning target volume (PTV) and sparing organ at risk structures (OARs).

Furthermore, knowledge-based planning (KBP) methods have been developed to help automate the process of treatment plan generation. KBP methods represent a data-driven approach to treatment planning whereby a database of preexisting clinical plans is utilized by predictive models to generate a new patient-specific plan. It involves the use of machine learning methods such as linear regression, principal component analysis, random forests, and neural networks to generate an initial plan which is then further optimized including manual input from the dosimetrist and physicians. Dose volume histogram (DVH) is a main metric used to characterize the dose distribution for given anatomical structures. The earlier KBP methods were dedicated to predicting the DVH using different underlying models/methods. DVH consists of zero-dimensional (such as mean/minimum/maximum dose) or one-dimensional metrics (volume-at-dose or dose-at-volume histograms) which lacks any spatial information. Methods based on learning to predict DVH statistics fail to take into account detailed voxel-level dose distribution in 2D or 3D. This shortcoming has led to a push towards development of methods for directly predicting voxel-level three-dimensional dose distributions.

A major driver in the push for predicting 3D voxel-level dose plans has been the advent of deep learning (DL) based methods. Originally developed for tasks such as natural image segmentation, object detection, image recognition, and speech recognition, deep learning methods have found applications in medical imaging, including radiation therapy. A DL dose prediction method uses a convolutional neural network (CNN) model which receives a 2D or 3D input in the form of planning CT with OAR/PTV masks and produces a voxel-level dose distribution as its output. The predicted dose is compared to the real dose using some form of loss function such as mean squared error, and gradients are backpropagated through the CNN model to iteratively improve the predictions. Many methods have been developed using different input configurations, with different network architectures and loss functions, and have been applied to various anatomical sites, including head and neck, prostate, pancreas, breast cancer, esophagus, and lung cancer sites. Another approach covers DL developments specifically for external beam radiotherapy automated treatment planning.

All the other DL-based methods, however, still rely on manually-generated plans for training. One approach demonstrated the importance of consistent training data on the performance of the DL model for esophageal cancer. This work compared the performance of the same model trained on variable as well as more homogeneous/consistent plan databases. The original database contained different machines, beam configurations, beam energies and involved different physicians and medical physicists for contouring and planning respectively, whereby the homogenized or consistent version was created by re-contouring, re-planning, and re-optimization of the plans done by the same observer with identical beam configurations. It was shown that a homogenized/consistent database led to higher performance compared to the original variable plan database. In the present disclosure, an automated treatment planning system (also referred to expedited constrained hierarchical optimization (ECHO)) may be used to generate consistent high-quality plans as an input for the DL model. ECHO generates consistent high-quality plans by solving a sequence of constrained large-scale optimization problem. ECHO is integrated with Eclipse and is used in the daily clinical routine, with more than 4000 patients treated to date. The integrated ECHO-DL system proposed in this work can be quickly adapted to the clinical changes using the complementary strengths of both the ECHO and DL modules, i.e., consistent/unbiased plans generated by ECHO and the fast 3D dose prediction by the DL module.

### 2. Materials and Method

### I. Patient Dataset

A database of 120 randomly selected lung cancer patients treated with conventional IMRT with 60 Gy in 30 fractions between the year 2018 and 2020 may be used. All these patients received treatment before clinical deployment of ECHO for lung disease site and therefore include the treated plans which were manually generated by planners using 5-7 coplanar beams and 6 MV energy. ECHO was run for these patients using the same beam configuration and energy. ECHO solves two constrained optimization problems where the critical clinical criteria in Table 1 are strictly enforced by using constraints, and PTV coverage and OAR sparing are optimized sequentially. ECHO can be run from Eclipse™ as a plug-in, and it typically takes 1-2 hours for ECHO to automatically generate a plan. ECHO extracts the data needed for optimization (e.g., influence matrix, contours) using Eclipse™ application programming interface (API), solves the resultant large-scale constrained optimization problems using commercial optimization engines (KNITRO™/AMPL™) and then imports the optimal fluence map into Eclipse for final dose calculation and leaf sequencing.

TABLE 1
Clinical Max/Mean dose (in Gy) and Dose-volume criteria

| Structure | Max (Gy) | Mean (Gy) | Dose-volume |
|---|---|---|---|
| PTV | 72 | | |
| Lungs Not GTV | 66 | 21 | V(20 Gy) <= 37% |
| Heart | 66 | 20 | V(30 Gy) <= 50% |
| Stomach | 54 | 30 | |
| Esophagus | 66 | 34 | |
| Liver | 66 | | V(30 Gy) <= 50% |
| Cord | 50 | | |
| Brachial Plexus | 65 | | |

### II. Inputs and Preprocessing

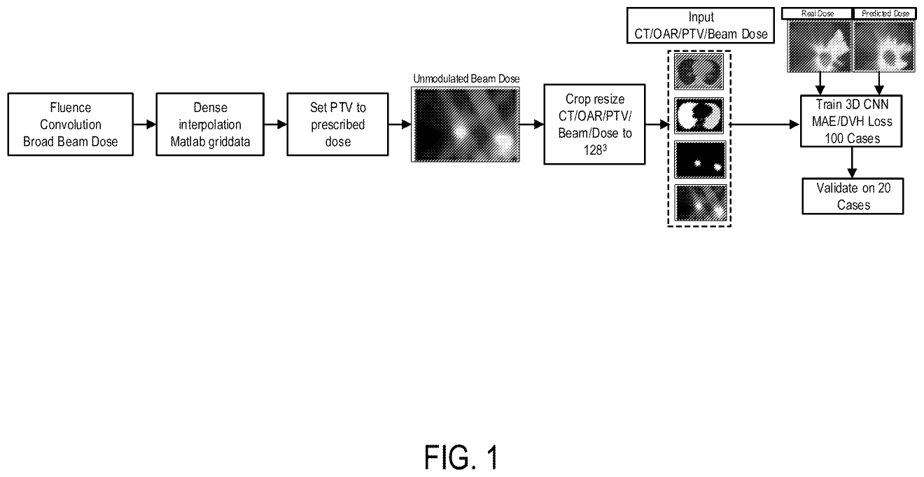

Structure contours and the 3D dose distribution corresponding to the treated manual plans and automated ECHO plans were extracted from the Eclipse V15.5 (Varian Medical Systems, Palo Alto, CA, USA). Each patient has a planning CT and corresponding PTV and OARs manual delineations which may differ from patient to patient depending on the location and size of the tumor. However, all patients have esophagus, spinal cord, heart, left lung, right lung and PTV delineated. Hence, these five OARs and PTV may be used as inputs in addition to the planning CT. The beam configuration information may be incorporated in the input using the fluence-convolution broad beam (FCBB) algorithm to generate an approximate dose distribution quickly. FIG. 1 shows the overall workflow to train a CNN to generate voxel-wise dose distribution.

The CT images may have different spatial resolutions but have the same in-plane matrix dimensions of 512×512. The PTV and OAR segmentation dimensions match those of the corresponding planning CTs. The intensity values of the input CT images are first clipped to have range of [−1000, 3071] and then rescaled to range [0, 1] for input to the DL network. The OAR segmentations are converted to a one-hot encoding scheme with value of 1 inside each anatomy and 0 outside. The PTV segmentation is then added as an extra channel to the one-hot encoded OAR segmentation.

The manual and ECHO dose data have different resolutions than the corresponding CT images. Each pair of the manual and ECHO doses is first resampled to match the corresponding CT image. The dose values are then clipped to values between [0, 70] Gy. For easier training and comparison between different patients, the mean dose inside PTV of all patients is rescaled to 60 Gy. This serves as a normalization for comparison between patients and can be easily shifted to a different prescription dose by a simple rescaling inside the PTV region. All the dose values inside the PTV may be set to the prescribed dose of 60 Gy and then resampled to match the corresponding CT, similar to the original manual/ECHO doses.

Finally, in order to account for the GPU RAM budget, a 300×300×128 region may be cropped from all the input matrices (CT/OAR/PTV/Dose/Beam configuration) and resampled to a consistent 128×128×128 dimensions. The OAR/PTV segmentation masks may be used to guide the cropping to avoid removing any critical regions of interest.

### III. CNN Architecture

A Unet-like CNN architecture may be trained to output the voxel-wise 3D dose prediction corresponding to an input comprising of 3D CT/contours and beam configuration all concatenated along the channel dimension. The network follows a common encoder-decoder style architecture which is composed of a series of layers which progressively downsample the input (encoder), until a bottleneck layer, where the process is reversed (decoder). Additionally, Unet-like skip connections are added between corresponding layers of encoder and decoder. This is done to share low-level information between the encoder and decoder counterparts.

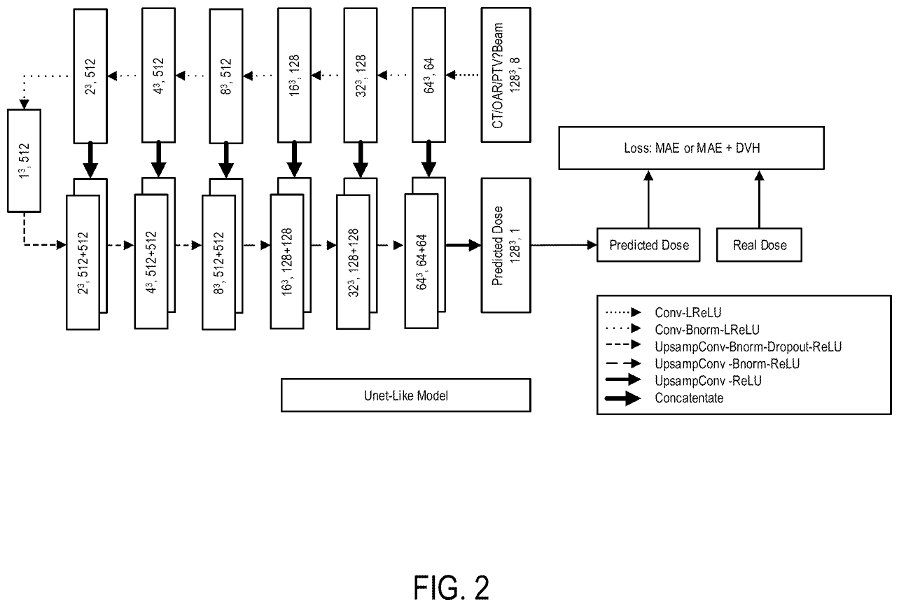

The network (FIG. 2) uses combinations of Convolution-BatchNorm-ReLU and Convolution-BatchNorm-Dropout-ReLU layers with some exceptions. Batchnorm is not used in the first layer of encoder, and all ReLU units in the encoder are leaky with slope of 0.2 while the decoder uses regular ReLU units. Whenever dropout is present, a dropout rate of 50% is used. All the convolutions in the encoder are 4×4×4 3D spatial filters with a stride of 2 in all 3 directions. The convolutions downsample by 2 in the encoder. In the decoder, trilinear upsampling may be used followed by regular 3×3×3 stride 1 convolution. The last layer in the decoder maps is input to a one channel output (1283, 1) followed by a ReLU non-linearity which gives the final predicted dose.

### IV. Loss Functions

Two types of loss functions may be used. First, mean absolute error (MAE) may be used as loss function which measures the error between paired observations, which are the real and predicted 3D dose. MAE is defined as

$$\frac{1}{N} {\sum }_{i} \vert {D}_{p} ( i ) - {D}_{r} ( i ) \vert$$

where N is the total number of voxels and Dₚ, Dᵣ are the predicted and real doses. MAE may be used versus a common alternative, mean squared error (MSE), as MAE produces less blurring in the output compared to MSE.

One approach has shown the importance of adding a domain-knowledge loss function based on DVH along with MAE. The non-differentiability issue of DVH can be addressed by approximating the heaviside step function by the readily differentiable sigmoid function. The volume-at-dose with respect to the dose dₜ is defined as the volume fraction of a given region-of-interest (OARs or PTV) which receives a dose of at least dₜ or higher. Given a segmentation mask, Mₛ, for the sth structure, and a volumetric dose distribution, D, the volume at or above a given threshold, dₜ, can be approximated as:

$${v}_{s , t} ( D , {M}_{s} ) = \frac{{\sum }_{i} σ ( \frac{D ( i ) - {d}_{t}}{β} ) {M}_{s} ( i )}{{\sum }_{i} {M}_{s} ( i )} \qquad ( 1 )$$

where σ is the sigmoid function,

$$σ ( x ) = \frac{1}{1 + {ℓ}^{- x}} , β$$

is histogram bin width, and i loops over the voxel indices of the dose distribution. The DVH loss can be calculated using MSE between the real and predicted dose DVH and is defined as follows:

$${L}_{DVH} ( {D}_{r} , {D}_{p} , M ) = \frac{1}{{n}_{s}} \frac{1}{{n}_{t}} {\sum }_{s} {‖ DVH ( {D}_{r} , {M}_{s} ) - DVH ( {D}_{p} , {M}_{s} ) ‖}_{2}^{2}$$

### V. Evaluation Criteria

To evaluate the quality of the predicted doses, the metrics used in a AAPM “open-access knowledge-based planning grand challenge” (OpenKBP) may be adopted. This competition was designed to advance fair and consistent comparisons of dose prediction methods for knowledge-based planning in radiation therapy research. The competition organizers used two separate scores to evaluate dose prediction models: dose score, which evaluates the overall 3D dose distribution, and a DVH score, which evaluates a set of DVH metrics. The dose score was simply the MAE between real dose and predicted dose. The DVH score that was chosen as a radiation therapy specific clinical measure of prediction quality involved a set of DVH criteria for each OAR and target PTV. Mean dose received by OAR was used as the DVH criteria for OAR while PTV had three criteria: D1, D95, and D99 which are the doses received by 1% (99^{th} percentile), 95% (5^{th} percentile), and 99% (1^{st} percentile) of voxels in the target PTV. DVH error, the absolute difference between the DVH criteria for real and predicted dose, was used to evaluate the DVHs. Average of all DVH errors was taken to encapsulate the different DVH criteria into a single score measuring the DVH quality of the predicted dose distributions.

Additional DVH metrics may also be reported for different anatomies typically used in clinical practice to evaluate dose plans. D2, D95, D98, D99 are radiation doses delivered to 2%, 95%, 98% and 99% of the volume and calculated as a percentage of the prescribed dose (60 Gy). Dmean (Gy) is the mean dose of the corresponding OAR again expressed as a percentage of prescribed dose. V5, V20, V35, V40 and V50 are the percentage of corresponding OAR volume receiving over 5 Gy, 20 Gy, 35 Gy, 40 Gy and 50 Gy respectively. The MAE (mean+STD) between the ground truth and predicted values of these metrics may be reported.

### VI. Deep Learning Settings

In these experiments, Stochastic Gradient Descent (SGD), with a batch size of 1, and Adam optimizer with an initial learning rate of 0.0002, and momentum parameters β1=0.5, β2=0.999, may be used to report the final results. The network may be trained for total of 200 epochs. A constant learning rate of 0.0002 may be used for the first 100 epochs, and then the learning rate may be let to linearly decay to 0 for the final 100 epochs. When using the MAE and DVH combined loss, the DVH component of the loss may be scaled by a factor of 10.

The training set of 100 images may be divided into train and validation set of 80 and 20 images respectively and the best learning rate and scaling factor for MAE+DVH loss may be determined. Afterwards, all the models may be trained using all 100 training datasets and tested on the holdout 20 datasets used for reporting results.

### 3. Results

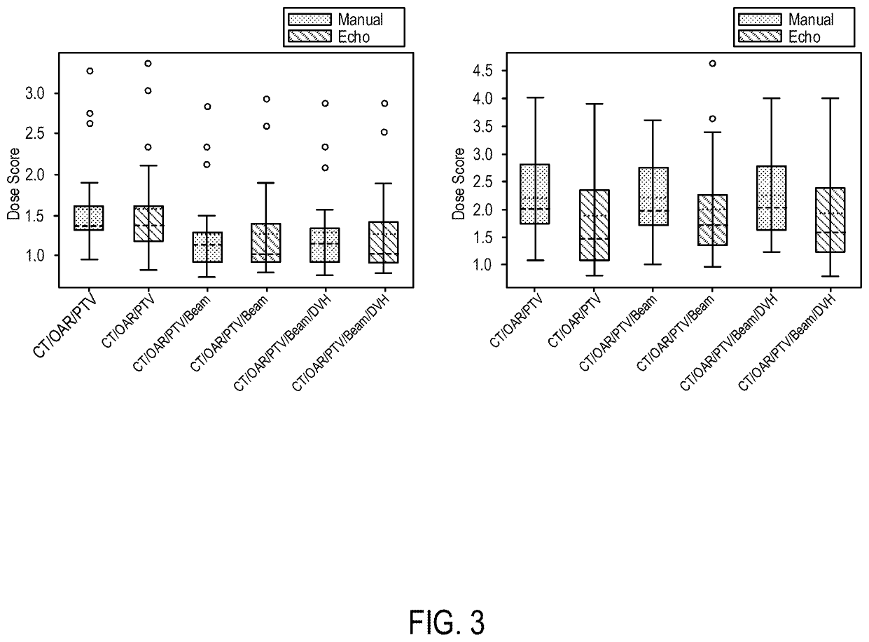

Table 2 presents the OpenKBP metrics for 3D dose prediction using ECHO and manual training data sets with different inputs ((1) CT+Contours and (2) CT+Contours+Beam) and different loss functions ((1) MAE and (2) MAE+DVH). The box plot of the metrics is also provided in FIG. 3 for better visual comparisons. DVH scores consistently show that the predictions for ECHO plans outperform the predictions for manual plans, whereas the dose scores show comparable results. Adding beam configuration seems to improve the dose-score for both ECHO and manual plans, while adding DVH loss function only benefits the DVH-score for ECHO plans.

TABLE 2
OpenKBP evaluation metrics for various experimental settings
including different inputs and loss functions to compare using
ECHO vs manual plans for dose prediction.

| | ECHO | | Manual | |
|---|---|---|---|---|
| | Dose Score | DVH Score | Dose Score | DVH Score |
| Experiment | (Gy) | (Gy) | (Gy) | (Gy) |
| CT + Contours + Beam/MAE + DVH | 1.27 ± 0.57 | 1.92 ± 0.92 | 1.29 ± 0.54 | 2.25 ± 0.73 |
| CT + Contours + Beam/MAE | 1.28 ± 0.59 | 1.99 ± 0.91 | 1.27 ± 0.54 | 2.21 ± 0.68 |
| CT + Contours/MAE | 1.58 ± 0.64 | 1.89 ± 1.02 | 1.59 ± 0.59 | 2.21 ± 0.79 |

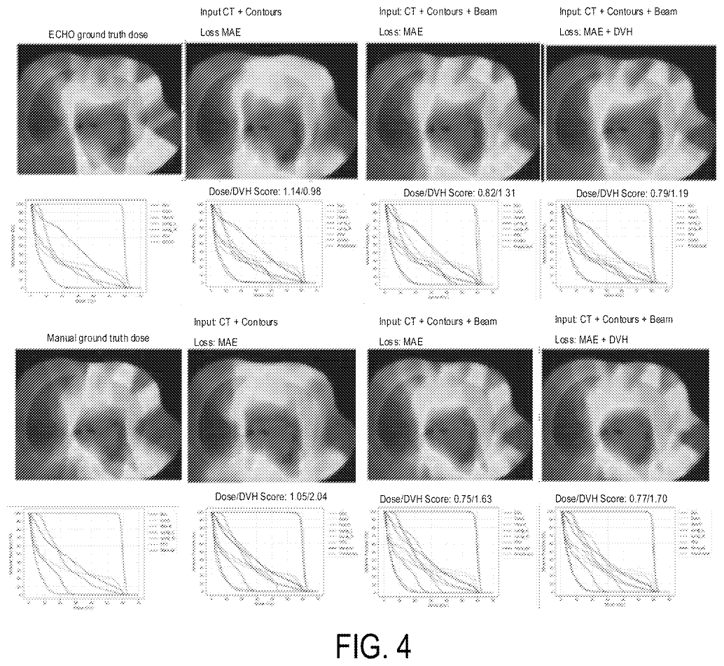

FIG. 4 shows an example of predicted manual and ECHO doses for the same patient using different input and loss function configurations. The dose distributions reveal the benefits of adding beams to the input. For both ECHO and manual plans, using only the CT, OAR and PTV as the input to the network produces generally blurred output dose. There is no visible dose spread in beam directions. Adding beam configuration as an extra input produces dose output which looks more like the real dose and spreads the dose more reliably along the beam directions. Without the beam configuration as an extra input, the DL network is unable to learn the beam structure and simply distributes the dose in the PTV and OAR regions. It has no concept of the physics of radiation beams and when the beam is used as an extra input, forcing the network to learn. DVH plots illustrate that adding DVH loss function slightly improves the ECHO prediction while it degrades performance for the manual plan prediction. Looking at both dose distribution and DVH, the best manual/ECHO results are obtained using all the inputs (CT+Contours+Beams), while adding DVH loss function only benefits ECHO.

Table 3 compares predictions of ECHO and manual plans using different configurations and clinically relevant metrics. Again, in general, the best result is obtained when the network is trained using ECHO plans with all the inputs and MAE+DVH as the loss function.

TABLE 3
Mean absolute error and its standard deviation (mean ± std) for
relevant DVH metrics on PTV and several organs for the test set
using manual and ECHO data with (a) CT + Contours/MAE, (b) CT +
Contours + Beam/MAE, and (c) CT + Contours + Beam/MAE +
DVH combinations. The values are expressed as percentage of
the prescription dose (Dₚᵣₑ = 60 Gy) for the metrics reporting the
dose received by x % of volume (Dₓ), and as an absolute difference
for the metrics reporting the volume (in %) receiving a dose of y Gy.

| | CT + Contours | CT + Contours + | CT + Contours + |
|---|---|---|---|
| | MAE | Beam MAE | Beam MAE + DVH |
| | ECHO | | |
| PTV | | | |
| D₉₉ (% of | 4.69 ± 6.98 | 3.85 ± 3.08 | 3.70 ± 3.57 |
| Dₚᵣₑ) | | | |
| D₉₈ (% of | 2.13 ± 2.71 | 3.31 ± 2.54 | 3.09 ± 2.95 |
| Dₚᵣₑ) | | | |
| D₉₅ (% of | 1.31 ± 1.40 | 2.46 ± 1.04 | 2.11 ± 1.80 |
| Dₚᵣₑ) | | | |
| D₅ (% of Dₚᵣₑ) | 1.15 ± 1.96 | 1.22 ± 1.02 | 1.51 ± 1.02 |
| Esophagus | | | |
| D₂ (% of Dₚᵣₑ) | 8.77 ± 10.25 | 8.53 ± 5.62 | 8.48 ± 5.31 |
| V₄₀ (% of | 8.78 ± 7.94 | 9.64 ± 9.24 | 10.58 ± 9.16 |
| volume) | | | |
| V₅₀ (% of | 16.28 ± 11.37 | 16.31 ± 12.16 | 16.16 ± 12.18 |
| volume) | | | |
| Heart | | | |
| V₃₅ (% of | 1.57 ± 1.43 | 2.18 ± 1.59 | 1.90 ± 1.71 |
| volume) | | | |
| Spinal Cord | | | |
| D₂ (% of Dₚᵣₑ) | 1.90 ± 2.82 | 2.11 ± 5.26 | 1.75 ± 3.97 |
| Left Lung | | | |
| Dₘₑₐₙ (% of | 1.91 ± 2.60 | 2.52 ± 4.03 | 2.44 ± 3.50 |
| Dₚᵣₑ) | | | |
| V₅ (% of | 4.08 ± 3.44 | 2.81 ± 2.30 | 2.61 ± 2.44 |
| volume) | | | |
| V₂₀ (% of | 3.53 ± 3.64 | 4.75 ± 3.49 | 4.67 ± 3.69 |
| volume) | | | |
| Right Lung | | | |
| Dₘₑₐₙ (% of | 2.55 ± 3.15 | 3.16 ± 3.10 | 2.95 ± 3.23 |
| Dₚᵣₑ) | | | |
| V₅ (% of | 0.01 ± 0.03 | 0.00 ± 0.02 | 0.01 ± 0.03 |
| volume) | | | |
| V₂₀ (% of | 0.01 ± 0.06 | 0.01 ± 0.03 | 0.01 ± 0.05 |
| volume) | | | |
| | Manual | | |
| PTV | | | |
| D₉₉ (% of Dₚᵣₑ) | 6.24 ± 4.95 | 6.68 ± 4.53 | 6.37 ± 4.93 |
| D₉₈ (% of Dₚᵣₑ) | 3.93 ± 3.23 | 4.84 ± 3.22 | 4.80 ± 3.42 |
| D₉₅ (% of Dₚᵣₑ) | 1.94 ± 1.84 | 2.96 ± 2.10 | 3.03 ± 2.19 |
| D₅ (% of Dₚᵣₑ) | 1.50 ± 1.20 | 1.52 ± 1.28 | 1.45 ± 1.29 |
| Esophagus | | | |
| D₂ (% of Dₚᵣₑ) | 11.33 ± 8.66 | 6.99 ± 5.26 | 7.44 ± 5.40 |
| V₄₀ (% of | 12.31 ± 8.90 | 9.34 ± 12.09 | 10.35 ± 11.83 |
| volume) | | | |
| V₅₀ (% of | 15.42 ± 16.03 | 15.46 ± 16.49 | 15.52 ± 16.47 |
| volume) | | | |
| Heart | | | |
| V₃₅ (% of | 2.44 ± 2.10 | 1.99 ± 1.75 | 1.98 ± 1.81 |
| volume) | | | |
| Spinal Cord | | | |
| D₂ (% of Dₚᵣₑ) | 1.14 ± 1.33 | 1.78 ± 2.96 | 2.01 ± 2.68 |
| Left Lung | | | |
| Dₘₑₐₙ (% of | 2.58 ± 1.92 | 2.64 ± 2.64 | 2.57 ± 2.44 |
| Dₚᵣₑ) | | | |
| V₅ (% of | 4.23 ± 2.84 | 3.76 ± 3.29 | 3.94 ± 3.70 |
| volume) | | | |
| V₂₀ (% of | 4.40 ± 3.60 | 5.27 ± 4.68 | 5.34 ± 4.71 |
| volume) | | | |
| Right Lung | | | |
| Dₘₑₐₙ (% of | 2.55 ± 2.24 | 3.05 ± 2.45 | 3.17 ± 2.45 |
| Dₚᵣₑ) | | | |
| V₅ (% of | 0.01 ± 0.03 | 0.00 ± 0.02 | 0.01 ± 0.03 |
| volume) | | | |
| V₂₀ (% of | 0.01 ± 0.06 | 0.01 ± 0.04 | 0.01 ± 0.05 |
| volume) | | | |

### 4. Discussion

This work shows an automated planning technique such as ECHO and a deep learning (DL) model for dose prediction can complement each other. The variability in the training data set generated by different planners can deteriorate the performance of deep learning models, and ECHO can address this issue by providing consistent high-quality plans. More importantly, offline-generated ECHO plans allow DL models to easily adapt themselves to changes in clinical criteria and practice. In addition, the fast predicted 3D dose distribution from DL models can guide ECHO to generate a deliverable Pareto optimal plan quickly; the inference time for the model is 0.4 seconds per case as opposed to 1-2 hours needed to generate the plan from ECHO.

One can use an unconstrained optimization framework and penalize the deviation of the delivered dose from the predicted dose to quickly replicate the predicted plan on the new patient. However, given the prediction errors, the lack of incentive to further improve the plan if possible, and the absence of constraints to ensure the satisfaction of important clinical criteria, the optimized plan may not be Pareto or clinically optimal. A more reliable and robust approach can leverage a constrained optimization framework such as ECHO. The predicted 3D dose can potentially accelerate the optimization process of solving large-scale constrained optimization problems by identifying and eliminating unnecessary/redundant constraints up front. For instance, a maximum dose constraint on a structure is typically handled by imposing the constraint on all voxels of that structure. Using 3D dose prediction, one can only impose constraints on voxels with predicted high doses and use the objective function to encourage lower doses to the remaining voxels. The predicted dose can also guide the patient's body sampling and reduce the number of voxels in optimization. For instance, one can use finer resolution in regions with predicted high dose gradient and coarser resolution otherwise.

The use of different inputs and loss functions that have been suggested by different groups may also be investigated. It was found that adding beams, along with CT and contours, improve the prediction for both manual and ECHO plans. For the loss function, however, this finding is only consistent with other groups when DL model is trained using ECHO plans (using MAE and DVH as loss function improves the prediction). This could be due to the large variation in manual plans!

### 5. Conclusion

This work highlights the impact of large variations in the training data on the performance of DL models for predicting 3D dose distribution. It also shows that the DL models can drastically benefit from an independent automated treatment planning system such as ECHO. The consistent and unbiased training data generated by ECHO not only enhances the prediction accuracy but can also allow the DL models to be rapidly and easily adapted to dynamic and constantly-changing clinical environments.

### B. Domain Knowledge-Driven 3D Dose Prediction Using Moment-Based Loss Function

Dose volume histogram (DVH) metrics are widely accepted evaluation criteria in the clinic. However, incorporating these metrics into deep learning dose prediction models is challenging due to their non-convexity and non-differentiability. Presented herein is a moment-based loss function for predicting 3D dose distribution for the challenging conventional lung intensity modulated radiation therapy (IMRT) plans. The moment-based loss function is convex and differentiable and can easily incorporate DVH metrics in any deep learning framework without computational overhead. The moments can also be customized to reflect the clinical priorities in 3D dose prediction. For instance, using high-order moments allows better prediction in high-dose areas for serial structures. A large dataset of 360 (240 for training, 50 for validation and 70 for testing) conventional lung patients with 2Gy×30 fractions may be used to train the deep learning (DL) model using clinically treated plans. A Unet-like CNN architecture may be trained using computed tomography (CT), planning target volume (PTV) and organ-at-risk contours (OAR) as input to infer corresponding voxel-wise 3D dose distribution. Three different loss functions may be used: (1) Mean Absolute Error (MAE) Loss, (2) MAE+DVH Loss, and (3) MAE+Moments Loss. The quality of the predictions was compared using different DVH metrics as well as dose-score and DVH-score, recently introduced by the AAPM knowledge-based planning grand challenge. Model with MAE+Moment loss function outperformed the MAE and MAE+DVH loss with DVH-score of 2.66±1.40 compared to 2.88±1.39 and 2.79±1.52 for the other two, respectively. Model with MAE+Moment loss also converged twice as fast as MAE+DVH loss, with training time of approximately 7 hours compared to 14 hours for MAE+DVH Loss. Sufficient improvement was found in D95 and D99 dose prediction error for PTV with better predictions for mean/max dose for OARs, especially cord and esophagus. Code and pretrained models will be released upon publication.

### 1. Introduction

Despite advances in optimization and treatment planning, intensity modulated radiation therapy (IMRT) treatment planning remains a time-consuming and resource-demanding task with the plan quality heavily dependent on the planner's experience and expertise. This problem is even more pronounced for challenging clinical cases such as conventional lung with complex geometry and intense conflict between the objectives of irradiating planning target volume (PTV) and sparing organ at risk structures (OARs). Balancing the trade-off between conflicting objectives can lead to sub-optimal plans, sacrificing the plan quality.

Several other techniques have been developed to automate or facilitate the radiotherapy treatment planning process. Multi-criteria optimization (MCO) facilitates the planning by generating a set of Pareto optimal plans upfront and allowing the user to navigate among them offline. Hierarchical constrained optimization enforces the critical clinical constraints using hard constraints and improves the other desirable criteria as much as possible by sequentially optimizing these. Knowledge-based planning (KBP) is a data-driven approach to automate the planning process by leveraging a database of pre-existing patients and learning a map between the patient anatomical features and some dose distribution characteristics. The earlier KBP methods used machine learning methods such as linear regression, principal component analysis, random forests, and neural networks to predict DVH as a main metric to characterize the dose distribution. However, DVH lacks any spatial information and only predicts dosage for the delineated structures.

Deep learning (DL) methods have been successfully used in radiation oncology for automated image contouring/segmentation as well as 3D voxel-level dose prediction. A DL dose prediction method uses a convolutional neural network (CNN) model which receives a 2D or 3D input in the form of planning CT with OAR/PTV masks and produces a voxel-level dose distribution as its output. The predicted dose is compared to the real dose using some form of loss function such as mean absolute error (MAE) or Mean square Error (MSE). The loss function in fact quantifies the goodness of the prediction by comparing it to the delivered dose voxel-by-voxel. While MAE and MSE are powerful and easy-to-use loss functions, they fail to integrate any domain-specific knowledge about the quality of dose distribution, including maximum/mean dose at each structure. The direct representation of DVH results in a discontinuous, non-differentiable, and non-convex function, which makes it difficult to integrate it into any DL model. One approach proposed a continuous and differentiable, yet non-convex, DVH-based loss function (not to be confused with predicting DVH).

Presented herein is a differentiable and convex surrogate loss function for DVH using multiple moments of dose distribution. Moments can approximate a DVH to an arbitrary accuracy, and also moments have been successfully used to replicate a desired DVH. The convexity and differentiability of the moment-based loss function makes it computationally appealing and also less prone to the local optimality. Furthermore, using different moments for different structures allows the DL model to drive the prediction according to the clinical priorities.

### 2. Materials and Method

### I. Loss Functions

Three types of loss functions may be used. First, mean absolute error (MAE) that measures the error between paired observations of real and predicted 3D dose may be used. MAE is defined as

$$\frac{1}{N} {\sum }_{i} \vert {D}_{p} ( i ) - {D}_{r} ( i ) \vert$$

where N is the total number of voxels and Dₚ, Dᵣ are the predicted and real doses. MAE may be used versus an alternative, mean squared error (MSE), as MAE produces less blurring in the output compared to MSE.

### II. Sigmoid-Based DVH Loss

One approach proposed approximating the heaviside step function by the readily differentiable sigmoid function to address discontinuity and non-differentiability issues of DVH function. For a given volumetric dose distribution D and a segmentation mask Mₛ for the sth structure, the volume-at-dose with respect to the dose di, denoted by V_{s,t}/(D, Mₛ), is defined as the volume fraction of a given region-of-interest (OARs or PTV) which receives a dose of at least di or higher which can be approximated as:

$${v}_{s , t} ( D , {M}_{s} ) = \frac{{\sum }_{i} σ ( \frac{D ( i ) - {d}_{t} {M}_{s} ( i )}{{\sum }_{i} {M}_{s} ( i )} )}{{\sum }_{i} {M}_{s} ( i )} \qquad ( 1 )$$

where σ is the sigmoid function,

$$σ ( x ) = \frac{1}{1 + {e}^{- x}} , β$$

is histogram bin width, and i loops over the voxel indices of the dose distribution. The DVH loss can be calculated using MSE between the real and predicted dose DVH and is defined as follows:

$${L}_{DVH} ( {D}_{r} , {D}_{p} , M ) = \frac{1}{{n}_{s}} \frac{1}{{n}_{t}} {\sum }_{s} {‖ DVH ( {D}_{r} , {M}_{s} ) - DVH ( {D}_{p} , {M}_{s} ) ‖}_{2}^{2} \qquad ( 2 )$$

### III. Moment Loss

Moment loss is based on the idea that a DVH can be well-approximated using a few moments:

$$DVH ∼ ( {M}_{1} , {M}_{2} , {M}_{3} , … , {M}_{p} )$$

where Mₚ represents the moment of order p defined as:

$${M}_{p} = {( \frac{1}{\vert {V}_{s} \vert} {\sum }_{j ∈ {V}_{s}} {d}_{j}^{p} )}^{\frac{1}{p}} \qquad ( 3 )$$

where Vₛ is a set of voxels belonging to the structure s, and dis the dose. M₁ is simply the mean dose of a structure whereas M_{∞} represents the max dose, and for p>1, Mₚ represents a value between mean and max doses.

In these experiments, a combination of three moments P={1, 2, 10} may be used for the critical OARs and PTV, where M₁ is exactly the mean dose, M₂ is the dose somewhere between the mean and max dose and Mio approximates the max dose.

The moment loss is calculated using mean square error between the actual and predicted moment for the structure:

$${L}_{moment} = {\sum }_{p ∈ P} {‖ {M}_{p} - {\overline{M}}_{p} ‖}_{2}^{2} \qquad ( 4 )$$

where Mₚ and M̅ₚ are the p^{th} moment of the actual dose and the predicted dose of a given structure, respectively.

### IV. Patient Dataset

360 randomly selected lung cancer patients treated with conventional IMRT with 60Gy in 30 fractions between the year 2017 and 2020 may be used. All these patients received treatment and therefore the treatment included the treated plans which were manually generated by experienced planners using 5-7 coplanar beams and 6 MV energy. Table 1 refers to the clinical criteria used. All these plans were generated using Eclipse™ V13.7-V15.5 (Varian Medical Systems, Palo Alto, CA, USA).

TABLE 1
Clinical Max/Mean dose (in Gy) and Dose-volume criteria

| Structure | Max (Gy) | Mean (Gy) | Dose-volume |
|---|---|---|---|
| PTV | 72 | | |
| Lungs Not GTV | 66 | 21 | V(20 Gy) <= 37% |
| Heart | 66 | 20 | V(30 Gy) <= 50% |
| Stomach | 54 | 30 | |
| Esophagus | 66 | 34 | |
| Liver | 66 | | V(30 Gy) <= 50% |
| Cord | 50 | | |
| Brachial Plexus | 65 | | |

### V. Inputs and Preprocessing

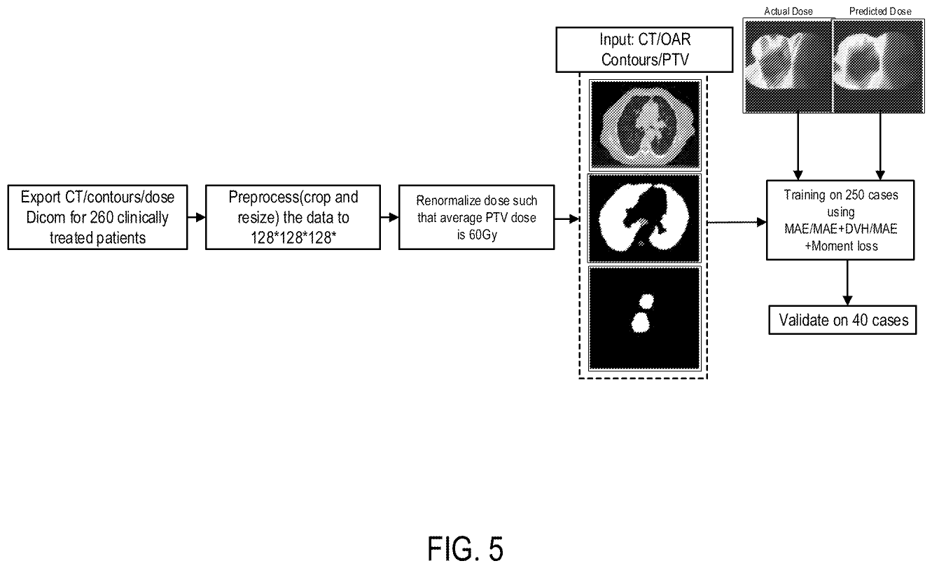

Structure contours and the 3D dose distribution were extracted from the Eclipse V15.5 (Varian Medical Systems, Palo Alto, CA, USA). Each patient has a planning CT and manually delineated contours of PTV and OARs which may differ from patient to patient depending on the location and size of the tumor. However, all patients have esophagus, spinal cord, heart, left lung, right lung and PTV delineated. Hence, these five OARs and PTV as inputs may be used in addition to the planning CT. FIG. 5 shows the overall workflow to train a CNN to generate voxel-wise dose distribution. The CT images may have different spatial resolutions but have the same in-plane matrix dimensions of 512×512. The PTV and OAR segmentation dimensions match those of the corresponding planning CTs. The intensity values of the input CT images are first clipped to have a range of [−1024, 3071] and then rescaled to range [0, 1] for input to the DL network. The OAR segmentations are converted to a one-hot encoding scheme with value of 1 inside each anatomy and 0 outside. The PTV segmentation is then added as an extra channel to the one-hot encoded OAR segmentation.

The dose data have different resolutions than the corresponding CT images. Each pair of the doses is first resampled to match the corresponding CT image. The dose values are then clipped to values between [0, 70] Gy. For easier training and comparison between different patients, the mean dose inside PTV of all patients is rescaled to 60 Gy. This serves as a normalization for comparison between patients and can be easily shifted to a different prescription dose by a simple rescaling inside the PTV region. All the dose values inside the PTV may be set to the prescribed dose of 60 Gy and then resampled to match the corresponding CT, similar to the original doses.

Finally, in order to account for the GPU RAM budget, a 300×300×128 region may be cropped from all the input matrices (CT/OAR/PTV/Dose/Beam configuration) and resampled to a consistent 128×128×128 dimensions. The OAR/PTV segmentation masks may be used to guide the cropping to avoid removing any critical regions of interest.

### VI. CNN Architecture

Unet is a fully connected network which has been widely used in the medical image segmentation. A Unet-like CNN architecture may be used to output the voxel-wise 3D dose prediction corresponding to an input comprising of 3D CT/contours which are concatenated along the channel dimension. The network follows an encoder-decoder style architecture which is composed of a series of layers which progressively downsample the input (encoder) using max pooling operation, until a bottleneck layer, where the process is reversed (decoder). Additionally, Unet-like skip connections are added between corresponding layers of encoder and decoder. This is done to share low-level information between the encoder and decoder counterparts.

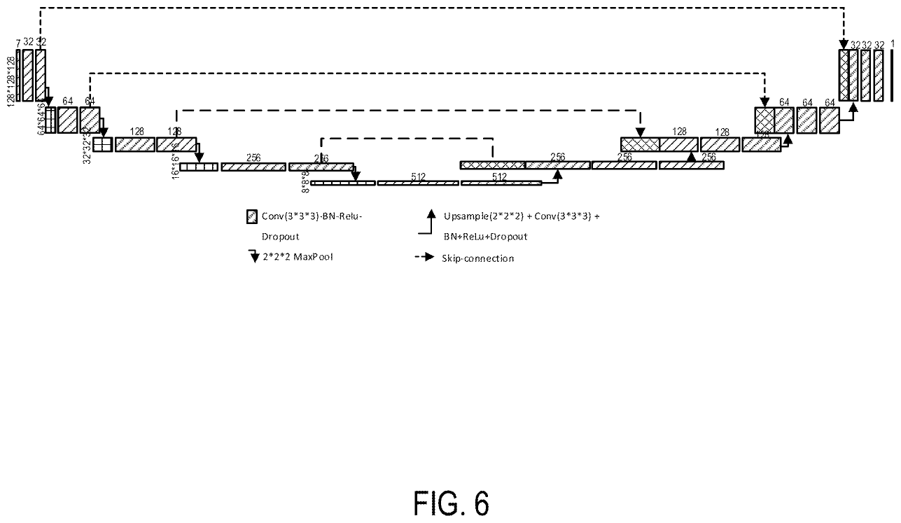

The network (FIG. 6) uses Convolution-BatchNorm-ReLU-Dropout as a block to perform series of convolution. Dropout is used with a dropout rate of 50%. Maxpool is used to downsample the image by 2 in each spatial level of encoder. All the convolutions in the encoder are 3×3×3 3D spatial filters with a stride of 1 in all 3 directions. In the decoder, trilinear upsampling may be used followed by regular 2×2×2 stride 1 convolution. The last layer in the decoder maps its input to a one channel output (128³, 1).

### VII. Evaluation Criteria

To evaluate the quality of the predicted doses, the metrics used in AAPM “open-access knowledge-based planning grand challenge” (OpenKBP) may be adopted. This competition was designed to advance fair and consistent comparisons of dose prediction methods for knowledge-based planning in radiation therapy research. The competition organizers used two separate scores to evaluate the dose prediction model. The dose score, which evaluates the overall 3D dose distribution and a DVH score, which evaluates a set of DVH metrics. The dose score was simply the MAE between real dose and predicted dose. The DVH score, which was chosen as a radiation therapy specific clinical measure of prediction quality, involved a set of DVH criteria for each OAR and target PTV. Mean dose received by OAR was used as the DVH criteria for OAR while PTV had two criteria: D95 and D99 which are the doses received by 95% (5^{th} percentile) and 99% (15% percentile) of voxels in the target PTV. DVH error, the absolute difference between the DVH criteria for real and predicted dose, was used to evaluate the DVHs. Average of all DVH errors was taken to encapsulate the different DVH criteria into a single score measuring the DVH quality of the predicted dose distributions.

### VIII. Deep Learning Settings

In these experiments, Stochastic Gradient Descent (SGD), with a batch size of 1, and Adam optimizer with an initial learning rate of 0.0002, and momentum parameters B1=0.5, 62=0.999, may be used. The network may be trained for total of 200 epochs. A constant learning rate of 0.0002 may be used for the first 100 epochs and then the learning rate may be let to linearly decay to 0 for the final 100 epochs. When using the MAE and DVH combined loss, the DVH component of the loss may be scaled by a factor of 10. Also, when using the MAE and Moment combined loss, the weight of 0.01 may be used for moment loss based upon these validation results.

The training set of 290 images may be divided into train and validation set of 240 and 50 images respectively and determined the best learning rate and scaling factor for (MAE+DVH) loss and (MAE+Moment) loss. Afterwards, all these models may be trained using all 290 training datasets and tested on the holdout 70 datasets used for reporting results.

### 3. Results

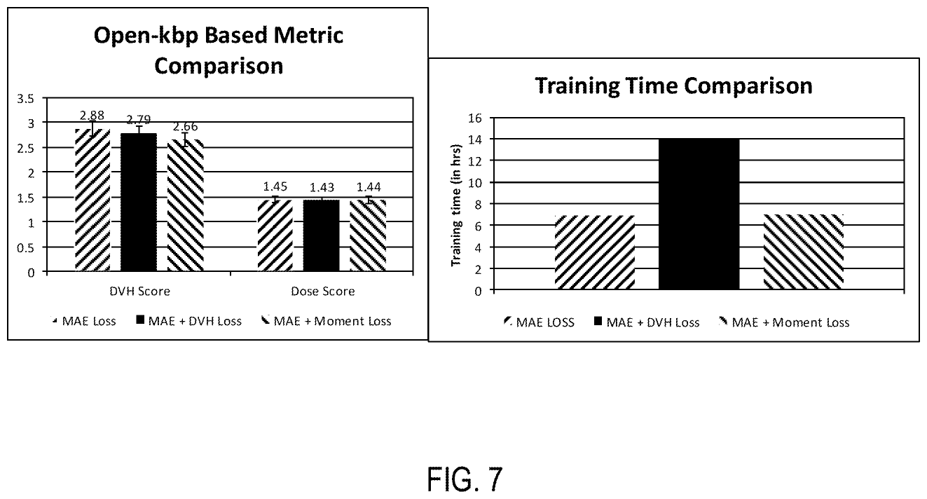

FIG. 7-left compares the results of three different loss functions (MAE loss, MAE+DVH loss and MAE+Moment loss) with respect to DVH-score and dose-score, introduced in Open-kbp challenge. DVH score of 2.66±1.40 for (MAE+Moment) loss outperformed DVH-score of 2.88±1.39 and 2.79±1.52 for the MAE and (MAE+DVH) loss, respectively. All models performed similarly with respect to the dose-score.

FIG. 7-right compares the three models with respect to their training time. MAE+DVH loss (˜14 hrs) is more time consuming due to its non-convexity and complex definition (see 1), while MAE+Moment loss is as efficient as MAE loss (˜7 hrs), owing to its convexity and simplicity (see 4).

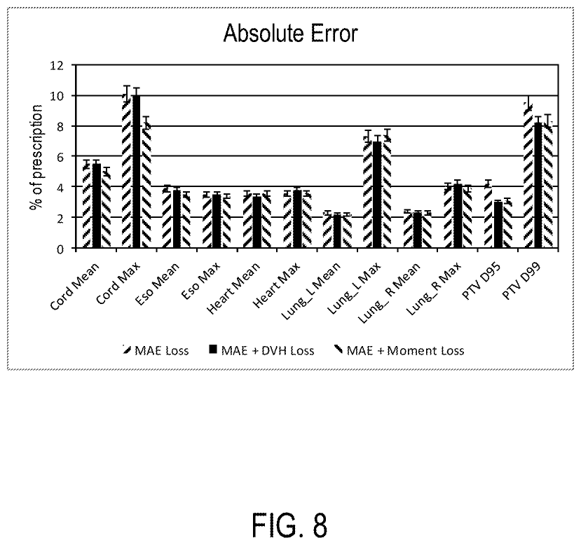

FIG. 8 shows the average absolute error between actual and predicted dose in terms of percentage of prescription for different clinically relevant criteria. Critical OARs like cord and esophagus showed substantial improvement in max/mean absolute dose error using (MAE+Moment) loss compared to other two in the category. PTV D95 and D99 showed marginal improvements in the dose prediction quality compared to MAE loss. There was small/no-improvement in the max/mean absolute error for other healthy organs (i.e., left lung, right lung, heart).

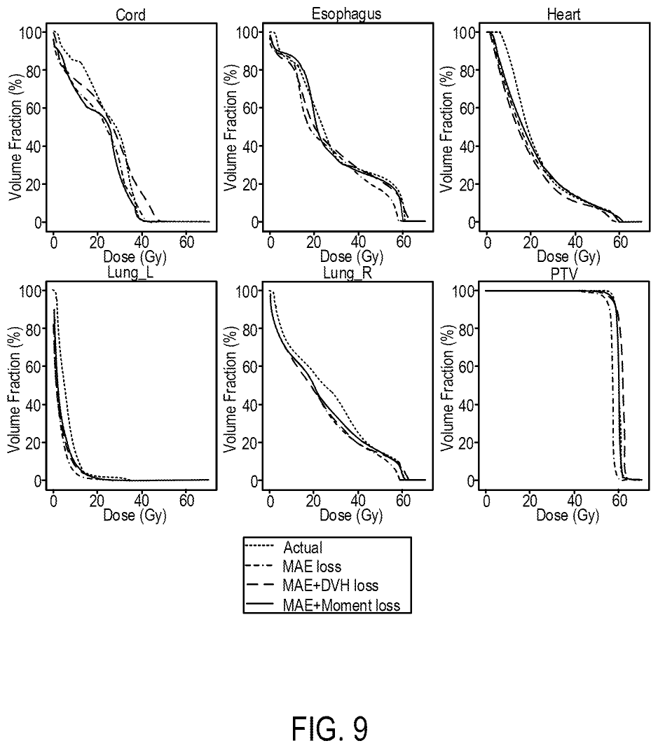

FIG. 9 compares the DVH of an actual dose (ground-truth here) with three predictions obtained from three different loss functions for a patient. As can been seen, in general, the prediction generated with (MAE+Moment) loss resembles the actual ground-truth dose more than the two other models.

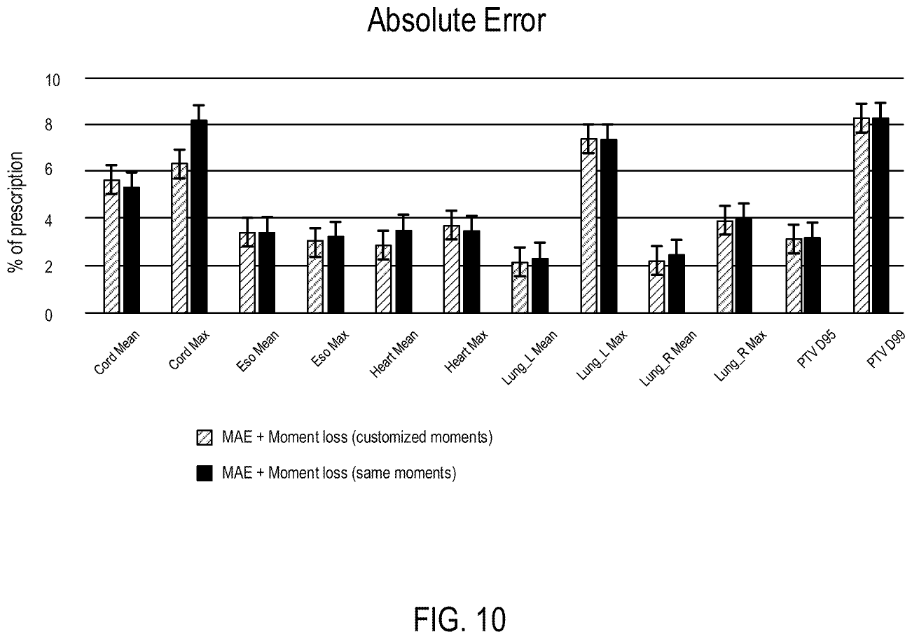

FIG. 10 shows the comparison of the absolute error for the model trained with default moments (p=1, 2, 10) for all the structures (red bar) and the model that used different moments for cord (p=5, 10) and heart (p=1, 2) and default moments (p=1, 2, 10) for all other structures (blue bar). As can be seen in the figure, using high-order moments for cord improves the maximum dose prediction, while using low-order moments for heart improves the mean dose prediction.

### 4. Discussion

Moments may be used as a surrogate loss function to integrate DVH into deep learning (DL) 3D dose prediction. Moments provide a mathematically rigorous and computationally efficient way to incorporate DVH information in any DL architecture without any computational overhead. This allows for incorporation of the domain-specific knowledge and clinical priorities into the DL model. Using MAE+Moment loss means the DL model tries to match the actual dose (ground-truth) not only at a micro-level (voxel-by-voxel using MAE loss) but also at a macro-level (structure-by-structure using representative moments).

Moments are essentially simple polynomial functions which can be calculated efficiently. Given their convexity, they do not suffer from the local optimality issue, making them more reliable choices with more robust behavior against the stochastic nature of the optimization techniques, commonly used in deep learning models. The computational efficiency of the moments allows training of large DL models. They also offer better fine-tuning of the hyper parameters.

The moments in conjunction with MAE help to incorporate DVH information into the DL model, however, the MAE loss still plays the central role in the prediction. In particular, the moments lack any spatial information about the dose distribution which is provided by the MAE loss. The MAE loss has also been successfully used across many applications and its performance is well-understood. Further research is needed to investigate the performance of the moment loss on more data especially with different disease sites.

The 3D dose prediction can facilitate and accelerate the treatment planning process by providing a reference plan which can be fed into a treatment planning optimization framework to be converted into a deliverable Pareto optimal plan. The dose-mimicking approach has been used, seeking the closest deliverable plan to the reference plan using quadratic function as a measure of distance. Another approach proposed an inverse optimization framework which estimates the objective weights from the reference plan and then generates the deliverable plan by solving the corresponding optimization problem. Any improvements in the prediction, including the ones using the proposed moment loss, needs to be ultimately evaluated using the entire pipeline of predicting a plan and converting that into a deliverable plan.

### 5. Conclusion

This work shows that the moments are powerful tools with sound mathematical properties to integrate DVH as an important domain knowledge into 3D dose prediction without any computational overhead. The idea has been validated on a large dataset of 360 challenging conventional lung patients.

### C. Systems and Methods of Determining Radiation Therapy Dosages to Administer to Subjects

Deep learning (DL) may be used to perform 3D dose prediction. However, the variability of plan quality in the training dataset, generated manually by planners with wide range of expertise, can dramatically affect the quality of the final predictions. Furthermore, any changes in the clinical criteria may result in a new set of manually generated plans by planners to build a new prediction model. To address these and other technical challenges, a computing system may establish and train machine learning (ML) models to use input biomedical images (e.g., tomograms) to automatically generate radiation therapy plans. In establishing the ML model, moment losses may be used to capture clinically relevant features for particular organs from which images are obtained and to encode these features in the ML model.

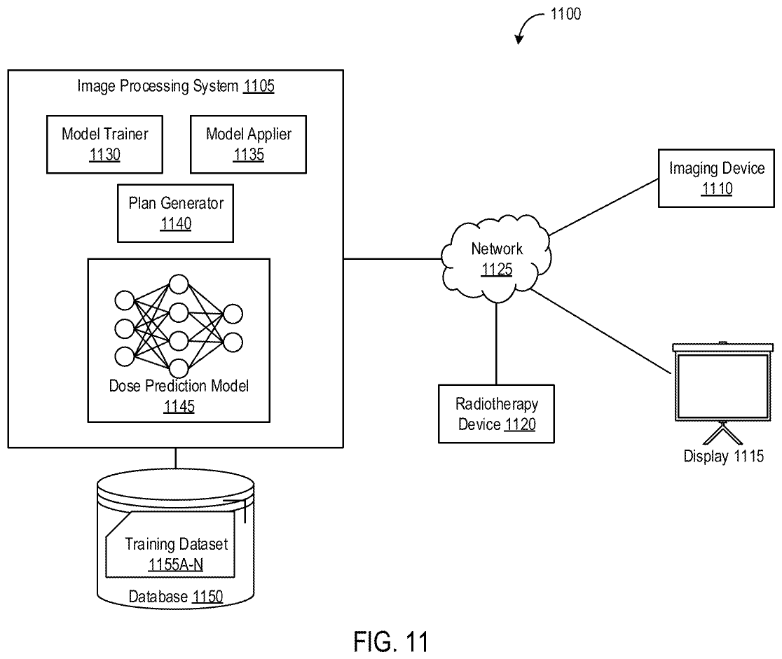

Referring now to FIG. 11, depicted is a block diagram of a system 1100 for determining radiation therapy dosages to administer. In overview, the system 1100 may at least one image processing system 1105, at least one imaging device 1110, and at least one display 1115, at least one radiotherapy device 1120, communicatively coupled with one another via at least one network 1125. The image processing system 1105 may include at least one model trainer 1130, at least one model applier 1135, at least one plan generator 1140, at least one dose prediction model 1145, and at least one database 1150. The database 1150 may include one or more training datasets 1155A-N(hereinafter generally referred to as training datasets 1155). Each of the components in the system 1100 as detailed herein may be implemented using hardware (e.g., one or more processors coupled with memory) or a combination of hardware and software as detailed herein in Section D. Each of the components in the system 1100 may implement or execute the functionalities detailed herein, such as those described in Sections A and B.

In further detail, the image processing system 1105 itself and the components therein, such as the model trainer 1130, the model applier 1135, and the dose prediction model 1145 may have a training mode and a runtime mode (sometimes herein referred to as an evaluation or inference mode). Under the training mode, the image processing system 1105 may invoke the model trainer 1130 to train the dose prediction model 1145 using the training dataset 1155. Under the runtime, the image processing system 1105 may invoke the model applier 1135 to apply the dose prediction model 1145 to acquired images from the imaging device 1110 and to provide radiotherapy plans to radiotherapy device 1120.

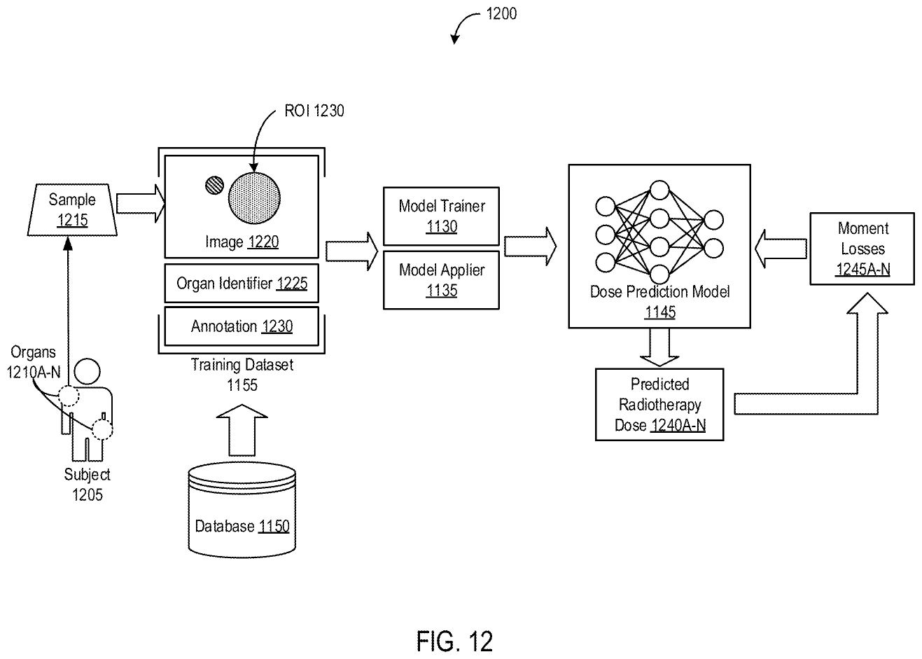

Referring now to FIG. 12, among others, depicted is a block diagram of a process 1200 to train models in the system 1100 for determining radiation therapy dosages to administer. The process 1200 may include or correspond to operations in the system 1100 for training the dose prediction model 1145 under the training mode. Under the process 1200, the model trainer 1130 executing on the image processing system 1105 may initialize or establish the dose prediction model 1145. The dose prediction model 1145 may have a set of weights (sometimes herein referred to as kernel parameters, kernel weights, or parameters). The set of weights may be arranged in a set of transform layers with one or more connections with one another to relate inputs and outputs of the dose prediction model 1145.

The architecture of the dose prediction model 1145 may be in accordance with an artificial neural network (ANN), such as one or more convolution neural networks (CNN). For instance, the dose prediction model 1145 may include the set of weights arranged across the set of layers according to the U-Net model detailed herein in conjunction with FIG. 2 or the encoder-decoder model detailed herein in conjunction with FIG. 6. Other architectures may be used for the dose prediction model 1145, such as an auto-encoder or a graph neural network (GNN), among others. In initializing, the model trainer 1130 may calculate, determine, or otherwise generate the initial values for the set of weights of the dose prediction model 1145 using pseudo-random values or fixed defined values.

The model trainer 1130 may retrieve, receive, or otherwise identify the training dataset 1155 to be used to train the dose prediction model 1145. In some embodiments, the model trainer 1130 may access the database 1150 to fetch, retrieve, or identify the one or more training datasets 1155. Each training dataset 1155 may correspond to an example radiation therapy plan previously created for a corresponding subject 1205 to treat a condition (e.g., a benign or malignant tumor) in at least one of organs 1210A-N (hereinafter generally referred to as organs 1210). The training dataset 1155 have been manually created and edited by a clinician examining the subject 1205 and at least one sample 1215 from the organ 1210 to be administered via radiotherapy. Each training dataset 1155 may identify or include at least one image 1220, at least one organ identifier 1225, at least one annotation 1230, among others.

The image 1220 (sometimes hereinafter referred to as a biomedical image or a tomogram) may be derived, acquired, or otherwise be of the sample 1215 of the subject 1205. For example, the image 1220 may be a scan of the sample 1215 corresponding to a tissue of the organ 1210 in the subject 1205 (e.g., human or animal). The image 1220 may include a set of two-dimensional cross-sections (e.g., a front, a sagittal, a transverse, or an oblique plane) acquired from the three-dimensional volume. The image 1220 may be defined in terms of pixels, in two-dimensions or three-dimensions. In some embodiments, the image 1220 may be part of a video acquired of the sample over time. For example, the image 1220 may correspond to a single frame of the video acquired of the sample over time at a frame rate.

The image 1220 may be acquired using any number of imaging modalities or techniques. For example, the image 1220 may be a tomogram acquired in accordance with a tomographic imaging technique, such as a magnetic resonance imaging (MRI) scanner, a nuclear magnetic resonance (NMR) scanner, X-ray computed tomography (CT) scanner, an ultrasound imaging scanner, and a positron emission tomography (PET) scanner, and a photoacoustic spectroscopy scanner, among others. The image 1220 may be a single instance of acquisition (e.g., X-ray) in accordance with the imaging modality, or may be part of a video (e.g., cardiac MRI) acquired using the imaging modality. Although primarily discussed in terms of a tomogram, other imaging modalities besides those listed above may be supported by the image processing system 1105 for the image 1220.

The image 1220 may include or identify at least one at least one region of interest (ROI) 1230 (also referred herein as a structure of interest (SOI) or feature of interest (FOI)). The ROI 1230 may correspond to an area, section, or part of the image 1220 that corresponds to feature in the sample 1215 from which the image 1220 is acquired. For example, the ROI 1230 may correspond to a portion of the image 1220 depicting a tumorous growth in a CT scan of a brain of a human subject. In some embodiments, the image 1220 may identify the ROI 1230 using at least one mask. The mask may define the corresponding area, section, or part of the image 1220 for the ROI 1230. The mask may be manually created by a clinician examining the image 1220 or may be automatically generated using an image segmentation tool to recognize the ROI 1230 from the image 1220.

In addition, the organ identifier 1225 may correspond to, reference, or otherwise identify the organ 1210 of the subject 1205 from which the sample 1215 is obtained for the corresponding image 1220. The organ identifier 1225 may, for example, be a set of alphanumeric characters identifying an organ type for the organ 1210 or an anatomical site for the sample 1215 taken form the organ 1210. The organ identifier 1225 may, for example, identify a brain, lung, heart, kidney, breast, prostate, ovary, pancreas, stomach, esophagus, bone, or an epidermis, among others, for any other type of organ 1210 of the subject 1205. The organ 1210 identified by the organ identifier 1225 may correspond to the anatomical site in the subject 1205 with the condition (e.g., tumorous cancer) to which radiation therapy is to be administered. The organ identifier 1225 may be manually created by a clinician examining the subject 1205 or the image 1220 or automatically generated by an image recognition tool to recognize which organ 1210 the image 1220 is obtained. In some embodiments, the organ identifier 1225 may be maintained using one or more files on the database 1150, separate from the files associated with the image 1220. In some embodiments, the organ identifier 1225 may be depicted within the image 1220 itself or included in metadata in the file of the image 1220.

For the image 1220 for each training dataset 1155, the annotation 1230 may identify, define, or otherwise include a radiation therapy dose to administer to the sample 1215 of the organ 1210 from the subject 1205. The annotation 1230 may include, for example, any number of parameters defining the radiation therapy expected to be administered to the sample 1215, such as an identification of a portion of the sample 1215 (or a set of voxels in the image 1220) to be administered with the radiotherapy dose; an intensity (or strength) of a radiation beam to be applied on the sample 1215; a shape of the radiation beam; a direction of the radiation beam relative to the sample 1215, and a duration (e.g., fractionation) of application of the beam on the sample 1215, among others. Depending on the type of organ 1210, the annotation 1230 may specify a mean dose intensity or a maximum dose intensity for the radiation beam to be applied. The annotation 1230 may have been manually created by a clinician examining the image 1220 derived from the sample 1215, the organ 1210 from which the sample 1215 is taken, and the subject 1205, among others. The annotation 1230 may be maintained using one or more files on the database 1150.

With the identification, the model applier 1135 may executing on the image processing system 1105 may apply or feed the image 1220 and the organ identifier 1225 from each training dataset 1155 to the dose prediction model 1145. The model applier 1135 may feed the image 1220 and the organ identifier 1225 from each training dataset 1155 as an input to the dose prediction model 1145. In feeding, the model applier 1135 may process the input image 1220 and the organ identifier 1225 in accordance with the set of weights of the dose prediction model 1145. The model applier 1135 may traverse through the set of training datasets 1155 to identify each input image 1220 and the organ identifier 1225 to feed into the dose prediction model 1145.

By processing, the model applier 1135 may output, produce, or otherwise generate at least one predicted radiotherapy dose 1240A-N(hereinafter generally referred to as a predicted radiotherapy dose 1240) for the input image 1220 and the organ identifier 1225 input into the dose prediction model 1145. From traversing over the set of training datasets 1155, the model applier 1135 may generate a corresponding set of predicted radiotherapy doses 1240. The set of predicted radiotherapy doses 1240 may be generated using images 1220 and organ identifiers 1225 from different subjects 1205, different organs 1210, and different samples 1215, among others, included in the training datasets 1155 on the database 1150.

Each predicted radiotherapy dose 1240 may be for the respective sample 1215 of the organ 1210 in the subject 1205 from which the input image 1220 is derived. The predicted radiotherapy dose 1240 may specify, define, or otherwise identify parameters defining the radiation therapy to be administered to the sample 1215, such as an identification of a portion of the sample 1215 (or a set of voxels in the image 1220) to be administered with the radiotherapy dose; an intensity (or strength) of a radiation beam to be applied on the sample 1215; a shape of the radiation beam; a direction of the radiation beam relative to the sample 1215, and a duration (e.g., fractionation) of application of the beam on the sample 1215, among others. Depending on the type of organ 1210, the predicted radiotherapy dose 1240 may identify a mean dose intensity or a maximum dose intensity for the radiation beam to be applied. The predicted radiotherapy dose 1240 may be in the form of one or more data structures (e.g., linked list, array, matrix, tree, or class object) outputted by the dose prediction model 1145.

The model trainer 1130 may calculate, determine, or otherwise generate one or more losses based on comparisons between the predicted radiotherapy doses 1240 and the corresponding annotations 1230 in the training datasets 1155. The losses may correspond to an amount of deviation between the predicted radiotherapy doses 1240 outputted by the dose prediction model 1145 and the expected radiotherapy doses as identified by the annotations 1230 in the training datasets 1155. In general, the higher the loss, the higher the deviation between the predicted and expected radiotherapy doses. Conversely, the lower the loss, the lower the deviation between the predicted and expected radiotherapy doses.

The model trainer 1130 may generate at least one moment loss 1245A-N (hereinafter generally referred to as moment loss 1245) for each organ 1210 (or other clinically relevant structure or parameter). The calculation of the moment losses 1245 may be in accordance with the techniques detailed herein in Sections A and B. To generate, the model trainer 1130 may determine a set of expected moments using the expected radiotherapy doses identified by the annotations 1230 in the training datasets 1155 for each organ 1210. In conjunction, the model trainer 1130 may determine a set of predicted moments using the predicted radiotherapy doses 1240 as outputted by the dose prediction model 1145 for each organ 1210. Each moment may identify, define, or otherwise correspond to a quantitative measure on a distribution of the expected radiotherapy doses for a given organ 1210. The moment may be any order, ranging from 0-th (e.g., corresponding to a mean dosage) to 10-th (e.g., corresponding to a maximum dosage), among others. In some embodiments, the determination of the set of expected and predicted moments may be further based on a set of voxels within each image 1220. The set of voxels may correspond to a portion of the sample 1215 of the organ 1210 to be applied with the expected or predicted radiotherapy dose.

With the determinations, the model trainer 1130 may calculate, determine, or otherwise generate the moment loss 1245 based on a comparison between the set of expected moments and the set of predicted moments. Each moment loss 1245 may be generated for a corresponding organ 1210. For instance, the model trainer 1130 may generate one moment loss 1245A using moments for the liver and another moment loss 1245B using moments for the lung across training datasets 1155 in the database 1150. The comparison may be between the expected moment and the corresponding predicted moment of the same order. The moment loss 1245 may be calculated in accordance with any number of loss functions, such as a norm loss (e.g., L1 or L2), mean squared error (MSE), a quadratic loss, a cross-entropy loss, and a Huber loss, among others.

In some embodiments, the model trainer 1130 may calculate, determine, or otherwise generate a voxel loss (sometimes herein referred to as mean absolute error). The voxel loss may reflect absolute discrepancy between the expected and predicted radiotherapy doses, independent of the type of organ 1210 or other clinically relevant parameters. The voxel loss may be based on a comparison between the predicted radiotherapy doses 1240 and the expected radiotherapy doses identified in the corresponding annotations 1230. For each training dataset 1155 inputted into the dose prediction model 1145, the model trainer 1130 may compare a set of voxels identified in the annotation 1230 to be applied with radiotherapy dose with a set of voxels identified in the predicted radiotherapy dose 1240. Based on the comparison, the model trainer 1130 may generate a voxel loss component for the input. Using the voxel loss components over all the inputs, the model trainer 1130 may generate the voxel loss. The voxel loss may be calculated in accordance with any number of loss functions, such as a norm loss (e.g., L1 or L2), mean squared error (MSE), a quadratic loss, a cross-entropy loss, and a Huber loss, among others.

With the determination, the model trainer 1130 may modify, change, or otherwise update at least one weights of the dose prediction model 1145. The updating of the weights of the dose prediction model 1145 using the losses, such as the moment losses 1245 for the corresponding set of organs 1210 and voxel losses over the training datasets 1155, among others. From updating using the moment losses 1245, the model train 1130 may encode clinically relevant latent parameters into the weights of the dose prediction model 1145. The updating of weights of the dose prediction model 1145 may be in accordance with an optimization function (or an objective function). The optimization function may define one or more rates or parameters at which the weights of the dose prediction model 1145 are to be updated. The updating of the weights of the dose prediction model 1145 may be repeated until convergence. Upon completion of training, the model trainer 1130 may store and maintain the set of weights of the dose prediction model 1145.

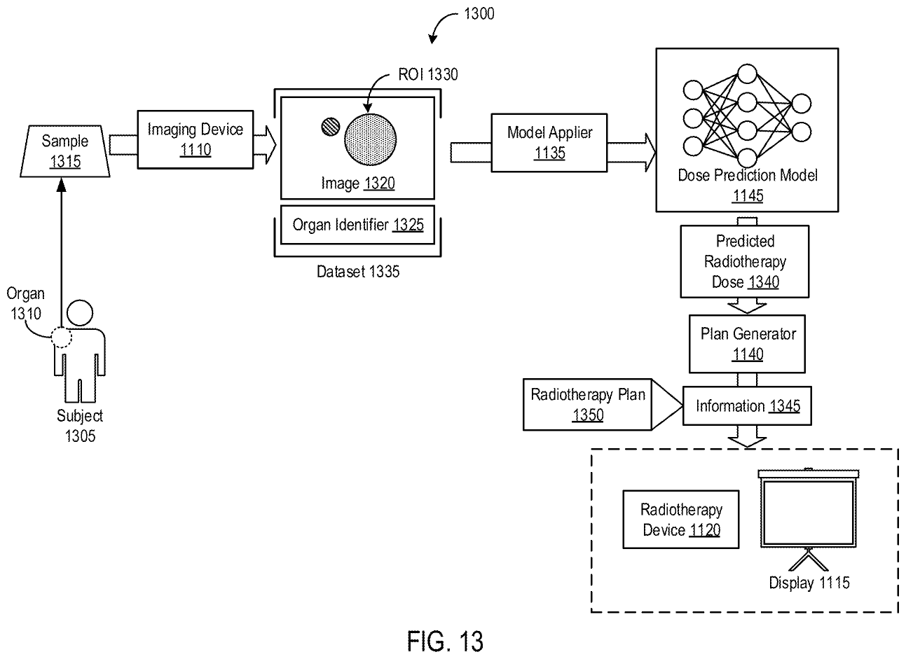

Referring now to FIG. 13, among others, depicted is a block diagram of a process 1300 for evaluating acquired images in the system 1100 for determining radiation therapy dosages to administer. The process 1300 may include or correspond to operations in the system 1100 under runtime or evaluation mode. Under the process 1300, the imaging device 1110 (sometimes herein referred to as an image acquirer) may produce, output, or otherwise generate at least one dataset 1335. The dataset 1335 may include or identify at least one image 1320 and at least one organ identifier 1325. The imagining device 1110 may generate the dataset 1335 in response to acquisition of the image 1320. The organ identifier 1325 may be manually inputted by a clinician examining the subject 1305 from which the sample 1315 of the organ 1310 is obtained for the image 1320.

The image 1320 (sometimes hereinafter referred to as a biomedical image or a tomogram) may be derived, acquired, or otherwise be of the sample 1315 of the subject 1305. The image 1320 may be acquired in a similar manner as image 1220 as discussed above. For example, the image 1320 may be a scan of the sample 1315 corresponding to a tissue of the organ 1310 in the subject 1305 (e.g., human or animal). The image 1320 may include a set of two-dimensional cross-sections (e.g., a front, a sagittal, a transverse, or an oblique plane) acquired from the three-dimensional volume. The image 1320 may be defined in terms of pixels, in two-dimensions or three-dimensions. In some embodiments, the image 1320 may be part of a video acquired of the sample over time. For example, the image 1320 may correspond to a single frame of the video acquired of the sample over time at a frame rate.

The image 1320 may be acquired using any number of imaging modalities or techniques. For example, the image 1320 may be a tomogram acquired in accordance with a tomographic imaging technique, such as a magnetic resonance imaging (MRI) scanner, a nuclear magnetic resonance (NMR) scanner, X-ray computed tomography (CT) scanner, an ultrasound imaging scanner, and a positron emission tomography (PET) scanner, and a photoacoustic spectroscopy scanner, among others. The image 1320 may be a single instance of acquisition (e.g., X-ray) in accordance with the imaging modality, or may be part of a video (e.g., cardiac MRI) acquired using the imaging modality. Although primarily discussed in terms of a tomogram, other imaging modalities besides those listed above may be supported by the image processing system 1105 for the image 1320.

The image 1320 may include or identify at least one at least one region of interest (ROI) 1330 (also referred herein as a structure of interest (SOI) or feature of interest (FOI)). The ROI 1330 may correspond to an area, section, or part of the image 1320 that corresponds to feature in the sample 1315 from which the image 1320 is acquired. For example, the ROI 1330 may correspond to a portion of the image 1320 depicting a tumorous growth in a CT scan of a brain of a human subject. In some embodiments, the image 1320 may identify the ROI 1330 using at least one mask. The mask may define the corresponding area, section, or part of the image 1320 for the ROI 1330. The mask may be manually created by a clinician examining the image 1320 or may be automatically generated using an image segmentation tool to recognize the ROI 1330 from the image 1320.

In addition, the organ identifier 1325 may correspond to, reference, or otherwise identify the organ 1310 (e.g., from a set of organs) of the subject 1305 from which the sample 1315 is obtained for the corresponding image 1320. The organ identifier 1325 may, for example, be a set of alphanumeric characters identifying an organ type for the organ 1310 or an anatomical site for the sample 1315 taken form the organ 1310. The organ identifier 1325 may, for example, identify a brain, lung, heart, kidney, breast, prostate, ovary, pancreas, stomach, esophagus, bone, or an epidermis, among others, for any other type of organ 1310 of the subject 1305. The organ 1310 identified by the organ identifier 1325 may correspond to the anatomical site in the subject 1305 with the condition (e.g., tumorous cancer) to which radiation therapy is to be administered. The organ identifier 1325 may be manually created by a clinician examining the subject 1305 or the image 1320 or automatically generated by an image recognition tool to recognize which organ 1310 the image 1320 is obtained. In some embodiments, the organ identifier 1325 may be maintained using one or more files on the database 1150, separate from the files associated with the image 1320. In some embodiments, the organ identifier 1325 may be depicted within the image 1320 itself or included in metadata in the file of the image 1320.

Upon acquisition and generation, the imaging device 1110 may send, transmit, or otherwise provide the dataset 1335 to the imaging processing system 1105. In some embodiments, the imaging device 1110 may send the datasets 1335 for a sample of a given subject 1310, organ 1310, or sample 1315, upon receipt of a request. The request may be received from the image processing system 1105 or another computing device of the user. The request may identify a type of sample (e.g., an organ or tissue) or the subject 1305 (e.g., using an anonymized identifier). In some embodiments, the imaging device 1110 may provide multiple datasets 1135 (e.g., for a given subject 1305) to the image processing system 1105.

The model applier 1135 may retrieve, identify, or otherwise receive the dataset 1335 from the imaging device 1110. With the identification, the model applier 1135 may executing on the image processing system 1105 may apply or feed the image 1320 and the organ identifier 1325 from the dataset 1335 to the dose prediction model 1145. The model applier 1135 may feed the image 1320 and the organ identifier 1325 as an input to the dose prediction model 1145. In feeding, the model applier 1135 may process the input image 1320 and the organ identifier 1325 in accordance with the set of weights of the dose prediction model 1145. The set of weights of the dose prediction model 1145 may be initialized, configured, or otherwise established in accordance with moment losses 1245 as discussed above. When multiple input datasets 1335 are provided, the model applier 1135 may traverse over the datasets 1335 to feed each dataset 1335 into the dose prediction model 1145.

By processing, the model applier 1135 may output, produce, or otherwise generate at least one predicted radiotherapy dose 1340 for the image 1320 and the organ identifier 1325 of the dataset 1335 input into the dose prediction model 1145. The predicted radiotherapy dose 1340 may specify, define, or otherwise identify parameters defining the radiation therapy to be administered to the sample 1315, such as an identification of a portion of the sample 1315 (or a set of voxels in the image 1320) to be administered with the radiotherapy dose; an intensity (or strength) of a radiation beam to be applied on the sample 1315; a shape of the radiation beam; a direction of the radiation beam relative to the sample 1315, and a duration of application of the beam on the sample 1315, among others. Depending on the type of organ 1310, the predicted radiotherapy dose 1340 may identify a mean dose intensity or a maximum dose intensity for the radiation beam to be applied. When multiple input datasets 1335 are provided, the model applier 1135 may generate a set of predicted radiotherapy doses 1340 from the dose prediction model 1145.

With the generation, the model applier 1135 may store and maintain the predicted radiotherapy dose 1340 for the subject 1305. In some embodiments, the model applier 1135 generate an association between the predicted radiotherapy dose 1340 and the dataset 1335. The association may be also with the subject 1305, the organ 1310, or the sample 1315 (e.g., using anonymized identifiers). The association may be in the form of one or more data structures (e.g., linked list, array, matrix, tree, or class object) outputted by the dose prediction model 1145. Upon generation, the model applier 1135 may store and maintain the association on the database 1150. In some embodiments, the model applier 1135 may provide the association to another computing device (e.g., communicatively coupled with the imaging device 1110 or display 1115).

The plan generator 1140 executing on the image processing system 1105 may produce or generate information 1345 based on the output predicted radiotherapy dose 1340 for the input dataset 1335. The information 1345 may identify, define, or otherwise include at least one radiotherapy plan 1350 for the subject 1305 to be administered with the radiotherapy. For example, the information 1345 may be a recommendation to a clinician examining the subject 1305. The information 1345 may identify or include parameters to carry out the predicted radiotherapy dose 1340, including the identification of a portion of the sample 1315 (or a set of voxels in the image 1320) to be administered with the radiotherapy dose; the intensity of the radiation beam; the shape of the beam; the direction of the radiation beam, and the duration of application, among others.

In some embodiments, the plan generator 1140 may generate the radiotherapy plan 1350 using one or more predicted radiotherapy doses 1340 outputted for the subject 1305. The plan generator 1140 may generate the radiotherapy plan 1350 based on the characteristics of the radiotherapy device 1120. The radiotherapy device 1120 may be, for instance, for delivering external beam radiation therapy (EBRT or XRT), sealed source radiotherapy, or unsealed source radiotherapy, among others. The radiotherapy device 1120 may be configured or controlled to carry out the radiotherapy plan 1350 to deliver the radiotherapy dose 1340 to the organ 1310 of the subject 1305. For example, for EBRT, the radiotherapy device 1120 may controlled to generate therapeutic X-ray beams of different strength, shape, direction, and duration, among other characteristics for the radiotherapy dose 1340. The information 1345 may include configuration parameters or commands to carry out the radiotherapy dose 1340 for the radiotherapy plan 1350. When multiple predicted radiotherapy doses 1340 are generated (e.g., for a given subject 1305), the plan generator 1140 may generate the radiotherapy plan 1350 as a combination of the predicted radiotherapy doses 1340. For instance, the radiotherapy plan 1350 may identify one predicted radiotherapy dose 1340 for one organ 1310 (e.g., the liver) and another predicted radiotherapy dose 1340 for another organ 1310 (e.g., the kidney) for a given subject 1305.

Upon generation, the plan generator 1140 may send, transmit, or otherwise provide the information 1345 associated with the predicted radiotherapy dose 1340. The information 1345 may be provided to the display 1115, the radiotherapy device 1120, or another computing device communicatively coupled with the image processing system 1105. The provision of the information 1345 may be in response to a request from a user of the image processing system 1105 or the computing device. The display 1115 may render, display, or otherwise present the information 1345, such as the subject 1305, the organ 1310, the image 1320, the predicted radiotherapy dose 1340, and the radiotherapy plan 1350, among others. For instance, the display 1115 may display, render, or otherwise present the information 1345 via a graphical user interface of an application to display predicted radiotherapy dose 1340 over the image 1320 depicting the organ 1310 within the subject 1305. The graphical user interface may be also used (e.g., by the clinician) to execute the radiotherapy plan 1350 via the radiotherapy device 1120. In addition, the radiotherapy device 1120 may execute the commands and other parameters of the radiotherapy plan 1350 upon provision.

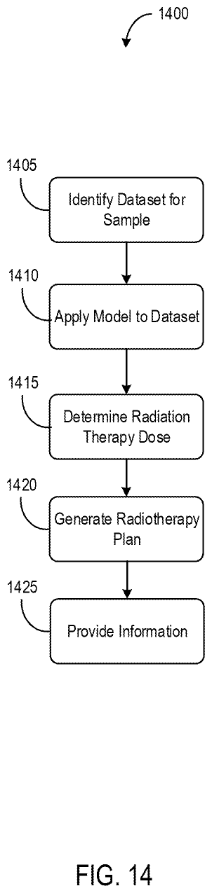

Referring now to FIG. 14, depicted is a flow diagram of a method 1400 of training models to determine radiation therapy dosages. The method 1400 may be performed by or implementing using the system 1100 described herein in conjunction with FIGS. 11-13 or the system 16 as described herein in conjunction with Section D. Under method 1400, a computing system (e.g., the image processing system 1105) may identify a dataset (e.g., the dataset 1335) for a sample (e.g., the sample 1315) (1405). The computing device may apply a model (e.g., the dose prediction model 1240) to the dataset (1410). The computing device may determine a radiation therapy dose (e.g., the predicted radiotherapy dose 1340) from the application (1415). The computing device may generate a radiotherapy plan (e.g., the radiotherapy plan 1350) using the determined radiation therapy dose (1420). The computing device may provide information (e.g., the information 1345) (1425).

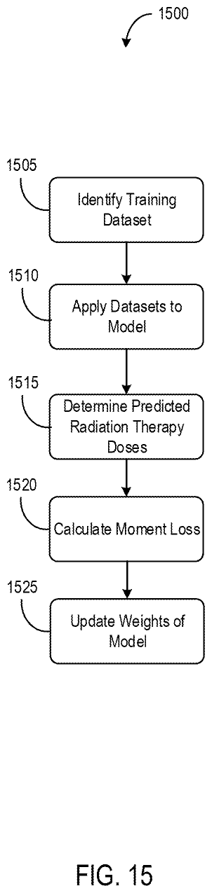

Referring now to FIG. 15, depicted is a flow diagram of a method 1500 of determining radiation therapy dosages. The method 1500 may be performed by or implementing using the system 1100 described herein in conjunction with FIGS. 11-13 or the system 16 as described herein in conjunction with Section D. Under method 1500, a computing system (e.g., the image processing system 1105) may identify a dataset (e.g., the training dataset 1155) for a sample (e.g., the sample 1215) (1505). The computing system may apply a model (e.g., the dose prediction model 1145) to the datasets (1510). The computing system may determine predicted radiotherapy doses (e.g., the predicted radiotherapy dose 1240) from application (1515). The computing system may calculate a moment loss (e.g., the moment loss 1245) for each organ (e.g., the organ 1210) (1520). The computing system may update weights of the model using the losses (1525).

### D. Computing and Network Environment

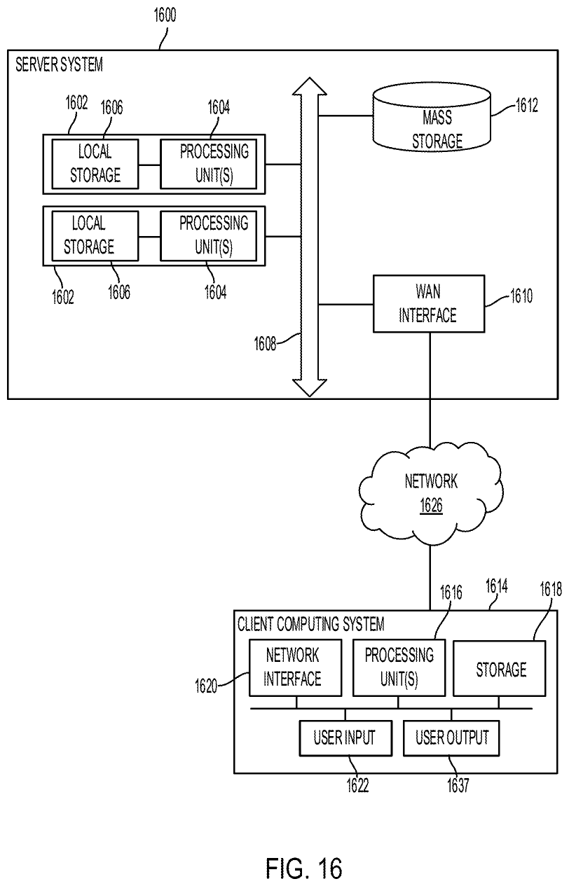

Various operations described herein can be implemented on computer systems. FIG. 16 shows a simplified block diagram of a representative server system 1600, client computer system 1614, and network 1626 usable to implement certain embodiments of the present disclosure. In various embodiments, server system 1600 or similar systems can implement services or servers described herein or portions thereof. Client computer system 1614 or similar systems can implement clients described herein. The systems 3700, 4200, and 4700 described herein can be similar to the server system 1600. Server system 1600 can have a modular design that incorporates a number of modules 1602 (e.g., blades in a blade server embodiment); while two modules 1602 are shown, any number can be provided. Each module 1602 can include processing unit(s) 1604 and local storage 1606.

Processing unit(s) 1604 can include a single processor, which can have one or more cores, or multiple processors. In some embodiments, processing unit(s) 1604 can include a general-purpose primary processor as well as one or more special-purpose co-processors such as graphics processors, digital signal processors, or the like. In some embodiments, some or all processing units 1604 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1604 can execute instructions stored in local storage 1606. Any type of processors in any combination can be included in processing unit(s) 1604.

Local storage 1606 can include volatile storage media (e.g., DRAM, SRAM, SDRAM, or the like) and/or non-volatile storage media (e.g., magnetic or optical disk, flash memory, or the like). Storage media incorporated in local storage 1606 can be fixed, removable, or upgradeable as desired. Local storage 1606 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1604 need at runtime. The ROM can store static data and instructions that are needed by processing unit(s) 1604. The permanent storage device can be a non-volatile read-and-write memory device that can store instructions and data even when module 1602 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1606 can store one or more software programs to be executed by processing unit(s) 1604, such as an operating system and/or programs implementing various server functions such as functions of the systems 3700, 4200, and 4700 or any other system described herein, or any other server(s) associated with systems 3700, 4200, and 4700 or any other system described herein.

“Software” refers generally to sequences of instructions that, when executed by processing unit(s) 1604, cause server system 1600 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1604. Software can be implemented as a single program or a collection of separate programs or program modules that interact as desired. From local storage 1606 (or non-local storage described below), processing unit(s) 1604 can retrieve program instructions to execute and data to process in order to execute various operations described above.

In some server systems 1600, multiple modules 1602 can be interconnected via a bus or other interconnect 1608, forming a local area network that supports communication between modules 1602 and other components of server system 1600. Interconnect 1608 can be implemented using various technologies including server racks, hubs, routers, etc.

A wide area network (WAN) interface 1610 can provide data communication capability between the local area network (interconnect 1608) and the network 1626, such as the Internet. Technologies can be used, including wired (e.g., Ethernet, IEEE 802.3 standards) and/or wireless technologies (e.g., Wi-Fi, IEEE 802.11 standards).

In some embodiments, local storage 1606 is intended to provide working memory for processing unit(s) 1604, providing fast access to programs and/or data to be processed while reducing traffic on interconnect 1608. Storage for larger quantities of data can be provided on the local area network by one or more mass storage subsystems 1612 that can be connected to interconnect 1608. Mass storage subsystem 1612 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1612. In some embodiments, additional data storage resources may be accessible via WAN interface 1610 (potentially with increased latency).

Server system 1600 can operate in response to requests received via WAN interface 1610. For example, one of modules 1602 can implement a supervisory function and assign discrete tasks to other modules 1602 in response to received requests. Work allocation techniques can be used. As requests are processed, results can be returned to the requester via WAN interface 1610. Such operation can generally be automated. Further, in some embodiments, WAN interface 1610 can connect multiple server systems 1600 to each other, providing scalable systems capable of managing high volumes of activity. Other techniques for managing server systems and server farms (collections of server systems that cooperate) can be used, including dynamic resource allocation and reallocation.

Server system 1600 can interact with various user-owned or user-operated devices via a wide-area network such as the Internet. An example of a user-operated device is shown in FIG. 16 as client computing system 1614. Client computing system 1614 can be implemented, for example, as a consumer device such as a smartphone, other mobile phone, tablet computer, wearable computing device (e.g., smart watch, eyeglasses), desktop computer, laptop computer, and so on.

For example, client computing system 1614 can communicate via WAN interface 1610. Client computing system 1614 can include computer components such as processing unit(s) 1616, storage device 1618, network interface 1620, user input device 1622, and user output device 1637. Client computing system 1614 can be a computing device implemented in a variety of form factors, such as a desktop computer, laptop computer, tablet computer, smartphone, other mobile computing device, wearable computing device, or the like.

Processor 1616 and storage device 1618 can be similar to processing unit(s) 1604 and local storage 1606 described above. Suitable devices can be selected based on the demands to be placed on client computing system 1614; for example, client computing system 1614 can be implemented as a “thin” client with limited processing capability or as a high-powered computing device. Client computing system 1614 can be provisioned with program code executable by processing unit(s) 1616 to enable various interactions with server system 1600.

Network interface 1620 can provide a connection to the network 1626, such as a wide area network (e.g., the Internet) to which WAN interface 1610 of server system 1600 is also connected. In various embodiments, network interface 1620 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1622 can include any device (or devices) via which a user can provide signals to client computing system 1614; client computing system 1614 can interpret the signals as indicative of particular user requests or information. In various embodiments, user input device 1622 can include any or all of a keyboard, touch pad, touch screen, mouse or other pointing device, scroll wheel, click wheel, dial, button, switch, keypad, microphone, and so on.

User output device 1637 can include any device via which client computing system 1614 can provide information to a user. For example, user output device 1637 can include display-to-display images generated by or delivered to client computing system 1614. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1637 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1604 and 1616 can provide various functionality for server system 1600 and client computing system 1614, including any of the functionality described herein as being performed by a server or client, or other functionality.

It will be appreciated that server system 1600 and client computing system 1614 are illustrative and that variations and modifications are possible. Computer systems used in connection with embodiments of the present disclosure can have other capabilities not specifically described here. Further, while server system 1600 and client computing system 1614 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including, but not limited to, specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished; e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media includes magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of determining radiation therapy dosages to administer, comprising:
identifying, by a computing system, a first dataset comprising: (i) a first biomedical image derived from a first sample to be administered with radiotherapy and (ii) a first identifier corresponding to a first organ of a plurality of organs from which the first sample is obtained;
applying, by the computing system, to the first dataset, a machine learning (ML) model comprising a plurality of weights trained using a plurality of second datasets in accordance with a moment loss for each of the plurality of organs, each of the plurality of second datasets comprising:
(i) a respective second biomedical image derived from a second sample,
(ii) a respective second identifier corresponding to a second organ of the plurality of organs from which the second sample is obtained, and
(iii) a respective annotation identifying a corresponding radiation therapy dose to administer to the second sample,
determining, by the computing system, from applying the first dataset to the ML model, a radiation therapy dose to administer to the sample from which the first biomedical image is derived; and
storing, by the computing system, using one or more data structures, an association between the first dataset and the radiation therapy dose.

2. The method of claim 1, further comprising providing, by the computing system, information for the radiotherapy to administer on the sample based on the association between the first dataset and the radiation therapy dose.

3. The method of claim 1, further comprising generating, by the computing system, a radiotherapy therapy plan to administer via a radiotherapy device to the first sample using the association between the first dataset and the radiation therapy dose.

4. The method of claim 1, wherein identifying the first dataset further comprises receiving a plurality of first datasets corresponding to a plurality of samples from one or more of the plurality of organs of the subject;
wherein determining the radiation therapy dose further comprises determining a plurality of radiation therapy doses to administer to the corresponding plurality of samples from one or more of the plurality of organs of the subject; and further comprising:
generating, by the computing system, a radiotherapy therapy plan to administer via a radiotherapy device to the subject based on the plurality of radiation therapy doses.

5. The method of claim 1, wherein determining the radiation therapy dose further comprises determining, from applying the first dataset to the ML model, at least one of a mean radiation therapy dose or a maximum radiation therapy dose based on the first organ.

6. The method of claim 1, wherein determining the radiation therapy dose further comprises determining, from applying the first dataset to the ML model, a plurality of parameters for the radiation therapy dose comprising one or more of: (i) an identification of a portion of the sample to be administered with the radiotherapy dose; (ii) an intensity of a beam to be applied on the first sample; (iii) a shape of the beam; (iv) a direction of a beam relative to the first sample, and (v) a duration of application of the beam on the first sample.

7. The method of claim 1, wherein the first biomedical image further comprises a first tomogram with a mask identifying a condition in a portion of the sample to be addressed via administration of the radiotherapy dose.

8. A method of training models to determine radiation therapy dosages to administer, comprising:
identifying, by a computing system, a plurality of datasets each comprising: (i) a respective biomedical image derived from a corresponding sample, (ii) a respective identifier corresponding to a respective organ of a plurality of organs from which the corresponding sample is obtained, and (iii) a respective annotation identifying a corresponding first radiation therapy dose to administer to the sample;
applying, by the computing system, to the plurality of datasets, a machine learning (ML) model comprising a plurality of weights to determine a plurality of second radiation therapy doses to administer;
generating, by the computing system, at least one moment loss for each organ of the plurality of organs based on a comparison between (i) a subset of the plurality of second radiation therapy for the organ and (ii) a corresponding set of first radiation therapy doses from a subset of the plurality of datasets each comprising the respective identified corresponding to the organ; and
modifying, by the computing system, one or more of the plurality of weights of the ML model in accordance with the at least one moment loss for each organ of the plurality of organs.

9. The method of claim 8, further comprising generating, by the computing system, a voxel loss based on (i) a second radiation therapy dose of the plurality of second radiation therapy doses and (ii) the corresponding first radiation therapy dose identified in the annotation;
wherein modifying the one or more of the plurality of weights further comprises modifying one or more of the plurality of weights of the ML model in accordance with a combination of the at least one moment loss for each organ and the voxel loss across the plurality of datasets.

10. The method of claim 8, wherein generating the at least one moment loss further comprises generating the at least one moment loss further based on a set of voxels identified for the organ within the respective biomedical image in at least one of the plurality of datasets.

11. The method of claim 8, wherein the ML model comprises the plurality of weights arranged in accordance with an encoder-decoder model to determine each of the plurality of second radiation therapy doses to administer using a corresponding dataset of the plurality of datasets.

12. The method of claim 8, wherein determining the plurality of second radiation therapy doses further comprises determining, from applying a dataset of the plurality of datasets to the ML model, at least one of a mean radiation therapy dose or a maximum radiation therapy dose based on the organ identified in the dataset.

13. The method of claim 8, wherein the first radiation therapy dose and the second radiation therapy dose each comprise one or more of: (i) an identification of a portion of the respective sample to be administered; (ii) an intensity of a beam to be applied; (iii) a shape of the beam; (iv) a direction of a beam, and (v) a duration of application of the beam.

14. The method of claim 8, wherein the respective biomedical image in each of the plurality of datasets further comprises a respective tomogram with a mask identifying a condition in a portion of the respective sample to be addressed via administration of the radiotherapy dose.

15. A system for determining radiation therapy dosages to administer, comprising:
a computing system having one or more processors coupled with memory, configured to:
identify a first dataset comprising: (i) a first biomedical image derived from a first sample to be administered with radiotherapy and (ii) a first identifier corresponding to a first organ of a plurality of organs from which the sample is obtained;
apply, to the first dataset, a machine learning (ML) model comprising a plurality of weights trained using a plurality of second datasets in accordance with a moment loss for each of the plurality of organs, each of the plurality of second datasets comprising:
(i) a respective second biomedical image derived from a second sample,
(ii) a respective second identifier corresponding to a second organ of the plurality of organs from which the second sample is obtained, and
(iii) a respective annotation identifying a corresponding radiation therapy dose to administer to the second sample,
determine, from applying the first dataset to the ML model, a radiation therapy dose to administer to the sample from which the first biomedical image is derived; and
store, using one or more data structures, an association between the first dataset and the radiation therapy dose.

16. The system of claim 15, wherein the computing system is further configured to provide information for the radiotherapy to administer on the sample based on the association between the first dataset and the radiation therapy dose.

17. The system of claim 15, wherein the computing system is further configured to generate a radiotherapy therapy plan to administer via a radiotherapy device to the first sample using the association between the first dataset and the radiation therapy dose.

18. The system of claim 15, wherein the computing system is further configured to:
receive a plurality of first datasets corresponding to a plurality of samples from one or more of the plurality of organs of the subject;
determine a plurality of radiation therapy doses to administer to the corresponding plurality of samples from one or more of the plurality of organs of the subject; and
generate a radiotherapy therapy plan to administer via a radiotherapy device to the subject based on the plurality of radiation therapy doses.

19. The system of claim 15, wherein the computing system is further configured to determine, from applying the first dataset to the ML model, a plurality of parameters for the radiation therapy dose comprising one or more of: (i) an identification of a portion of the sample to be administered with the radiotherapy dose; (ii) an intensity of a beam to be applied on the first sample; (iii) a shape of the beam; (iv) a direction of a beam relative to the first sample, and (v) a duration of application of the beam on the first sample.

20. The system of claim 15, wherein the first biomedical image further comprises a first tomogram with a mask identifying a condition in a portion of the sample to be addressed via administration of the radiotherapy dose.
